(19) United States
(12) Patent Application Publication
(10) Pub. No.: US 2025/0352761 A1
(43) Pub. Date: Nov. 20, 2025

(54) MEDICAL DEVICES WITH TUBULAR REINFORCEMENT

(71) Applicants: STRYKER CORPORATION, Portage, MI (US); STRYKER EUROPEAN OPERATIONS LIMITED, Carrigtwohill (IE)

(72) Inventors: Shana B. Castelli, San Francisco, CA (US); Michael Troy Poor, San Jose, CA (US); Lee Chen Puckett, Long Beach, CA (US); Murtagh M. Murphy, Cork (IE); Madalyn Diane Kern, South Salt Lake, UT (US)

(73) Assignees: STRYKER CORPORATION, Portage, MI (US); STRYKER EUROPEAN OPERATIONS LIMITED, Carrigtwohill (IE)

(21) Appl. No.: 19/292,201

(22) Filed: Aug. 6, 2025

Related U.S. Application Data
(63) Continuation of application No. PCT/US2024/059052, filed on Dec. 6, 2024.
(60) Provisional application No. 63/610,359, filed on Dec. 14, 2023.

(51) Int. Cl. A61M 25/00

(52) U.S. Cl. CPC A61M 25/0045; A61M 25/0052; A61M 25/0053; A61M 2025/0059; A61M 2205/0216

(57) ABSTRACT

A catheter includes: a tubular structure having a first ring element, a second ring element, and a third ring element; wherein the tubular structure comprises a first set of connecting members between the first ring element and the second ring element; wherein the tubular structure comprises a second set of connecting members between the second ring element and the third ring element; wherein the second ring element comprises a first ring portion and a second ring portion, wherein each of the first and second ring portions extends in a non-perpendicular direction with respect to a longitudinal axis of the tubular structure, and wherein the first and second rings together form a non-planar configuration for the second ring element.

### RELATED APPLICATION DATA

This application is a continuation of International Patent Application No. PCT/US2024/059052, filed on Dec. 6, 2024, which claims the benefit of U.S. Provisional Patent Application Ser. No. 63/610,359, filed on Dec. 14, 2023, the entire disclosures of all of which are hereby incorporated herein by reference in their entirety into the present application.

### FIELD

The present disclosure relates generally to minimally invasive medical devices, and more specifically to catheters.

### BACKGROUND

The use of intravascular catheters for accessing and treating various types of diseases, such as vascular defects, is well-known. For example, a suitable intravascular catheter may be inserted into the vascular system of a patient. Commonly used vascular application to access a target site in a patient involves inserting a guidewire (e.g., coronary guidewire, peripheral guidewire, neurovascular guidewire, etc.) through an incision in the femoral artery near the groin, advancing a guide-catheter and introducer/diagnostic catheter over the wire, and advancing both, with the guidewire tip leading, until the guide-catheter is placed in the common carotid or internal carotid arteries. The introducer and/or diagnostic catheter is then removed, and a neurovascular catheter is advanced through the guide-catheter and typically over a guidewire (and often with a microcatheter or delivery assist catheter as well) until the neurovascular catheter is disposed at the target site. Simultaneously or after placement of the distal end of the neurovascular catheter at the target site, any devices within the neurovascular catheter's lumen are removed, and a therapy is provided through the neurovascular catheter lumen, such as via injection of a drug, delivery of an implant, aspirating through the neurovascular catheter, etc.

For trackability, the flexibility of the distal segment of the catheter is critical. Currently, catheters have a solid markerband made from radiopaque material at the distal end of the catheter, which allows the catheter to be imaged. The solid markerband is a solid ring that joins to a distal end of a hypotube. The solid ring stiffens the catheter, making it difficult to track and to deflect away from branching arteries such as the ophthalmic artery. Thus, new marker device that reduces the stiffness of the catheter would be desirable.

Also, in certain applications, such as neurovascular treatment, the catheters are required to navigate tortuous and intricate vasculature. By using an appropriately sized device having the requisite performance characteristics, such as “pushability” “trackability” and most important, distal tip flexibility, virtually any target site in the vascular system may be accessed, including that within the tortuous cerebral, peripheral or venous vasculature. Further, the forces applied at the proximal end of these catheters should be transferred to the distal ends for suitable pushability (axial rigidity) and torquability (rotation). Achieving a balance between these features is highly desirable, but difficult.

In addition, a catheter may have a lumen with a certain cross-sectional shape. During use, the catheter may be bent. For example, tensioning wire may be operated to bend the catheter, and/or the catheter may be bent via guidewire or by a curvature of an anatomy. The bending of the catheter causes a compression on one side of the catheter and tension on an opposite side of the catheter. In some cases, due to the compression associated with the bending of the catheter, the catheter may kink, thereby collapsing the lumen of the catheter. Designing a catheter to resist such kinking while achieving certain bending flexibility and torsional rigidity is very difficult to accomplish.

### SUMMARY

A catheter includes: a tubular structure having a first ring element, a second ring element, and a third ring element; wherein the tubular structure comprises a first set of connecting members between the first ring element and the second ring element; wherein the tubular structure comprises a second set of connecting members between the second ring element and the third ring element; wherein the second ring element comprises a first ring portion and a second ring portion, wherein each of the first and second ring portions extends in a non-perpendicular direction with respect to a longitudinal axis of the tubular structure, and wherein the first ring portion and the second ring portion together form a non-planar configuration for the second ring element.

Optionally, the first ring portion and the second ring portion are different respective circumferential parts of the second ring element.

Optionally, the first ring portion has a first flat part lying in a first plane, wherein the second ring portion has a second flat part lying in a second plane that is different from the first plane, and wherein each of the first plane and the second plane forms a non-perpendicular angle with respect to a longitudinal axis of the catheter.

Optionally, the second ring element also comprises a joint portion between the first ring portion and the second ring portion, wherein the joint portion comprises a central portion, a first portion, and a second portion, and wherein the first and the second portions are on opposite sides of the central portion.

Optionally, the first portion and the second portion of the joint portion are curving in respective opposite directions.

Optionally, the central portion of the joint portion is configured to rotate and/or to resist a moment when the catheter is being tensioned or is undergoing bending.

Optionally, the first set of connecting members and the second set of connecting members are configured to move relative to the second ring element during bending and/or axial loading of the catheter.

Optionally, one of the connecting members in the first set has a width and a thickness, and wherein the width is less than the thickness.

Optionally, a total number N of the connecting members in the first set meets the condition N≥F/(S*A), where F is an axial load on the catheter, S is an allowable axial stress on each of the connecting members in the first set, and A is an area of a cross section of one of the connecting members.

Optionally, a space between one of the connecting members in the first set and the second ring element has a width L, and wherein the width L of the space meets the condition L<(nπ2EI/F)1/2, where n=4 or lower, E is an elastic modulus of a material of a polymeric structure spanning the space, F is an axial loading on the polymeric structure, and I is a moment of inertia of a cross section of the polymeric structure.

Optionally, a space between one of the connecting members in the first set and the second ring element has a width L, wherein the catheter further comprises a polymeric structure spanning the space, the polymeric structure having an elastic modulus E, and wherein the ratio E/L2 meets the below condition: F/(nπ2I)<E/L2, where F is an axial loading on the polymeric structure, n=4 or lower, and I is a moment of inertia of a cross section of the polymeric structure.

Optionally, the catheter further includes a marker element attached to or extending from a distal end of the tubular structure.

Optionally, the catheter further includes a polymer layer coupled to the tubular structure, wherein the polymer layer extends distally past a distal tip of the marker element to form a polymeric tip, wherein the polymeric tip extends less than 3 mm beyond the distal tip of the marker element.

Optionally, the catheter further includes a first polymer layer disposed circumferentially around an exterior surface of the tubular structure.

Optionally, the first polymer layer has an elastic modulus that is at least 2 Mpa and/or at most 7 Mpa, a tensile elongation (strain) at yield that is at least 300%, a tensile elongation (strain) at break point that is at least 500%, or any combination of the foregoing.

Optionally, the catheter further includes a second polymer layer disposed at an interior surface of the tubular structure.

Optionally, the second polymer layer has an elastic modulus that is at least 2 Mpa and/or at most 7 Mpa, a tensile elongation (strain) at yield that is at least 300%, a tensile elongation (strain) at break point that is at least 500%, or any combination of the foregoing.

Optionally, the first polymer layer and the second polymer layer form a tubing encapsulating the tubular structure; and wherein a material of the tubing has a modulus elasticity that is higher than a material of the tubular structure, and is more elastic than the material of the tubular structure.

Optionally, a stiffness of a combination of the tubular structure and the tubing is at least four times a stiffness of the tubular structure alone.

Optionally, the tubular structure has a wall gap to kerf width ratio that is anywhere from 1 to 5.

A catheter includes: a tubular structure having a first ring element, a second ring element, and a third ring element; wherein the tubular structure also comprises a first set of connecting members between the first ring element and the second ring element; wherein the tubular structure also comprises a second set of connecting members between the second ring element and the third ring element; wherein the second ring element comprises a first ring portion and a second ring portion, and wherein the first and second ring portions together form a non-planar configuration for the second ring element.

Optionally, the first ring portion and the second ring portion have a same configuration.

Optionally, the first ring portion and the second ring portion are different respective circumferential parts of the second ring element.

Optionally, wherein the first ring portion has a first flat part lying in a first plane, wherein the second ring portion has a second flat part lying in a second plane that is different from the first plane, and wherein each of the first plane and the second plane forms a non-perpendicular angle with respect to a longitudinal axis of the catheter.

Optionally, the second ring element also comprises a joint portion between the first ring portion and the second ring portion.

Optionally, the joint portion comprises a central portion, a first portion, and a second portion, and wherein the first and second portions are on opposite sides of the central portion.

Optionally, the first portion and the second portion of the joint portion are curving in respective opposite directions.

Optionally, the central portion of the joint portion is configured to rotate and/or to resist a moment when the catheter is being tensioned or is undergoing bending.

Optionally, the first set of connecting members and the second set of connecting members are configured to move relative to the second ring element during bending and/or axial loading of the catheter.

Optionally, one of the connecting members in the first set has a width and a thickness, and wherein the width is less than the thickness.

Optionally, a total number N of the connecting members in the first set meets the condition N≥F/(S*A), where F is an axial load on the catheter, S is an allowable axial stress on each of the connecting members in the first set, and A is an area of a cross section of one of the connecting members.

Optionally, a space between one of the connecting members in the first set and the second ring element has a width L, and wherein the width L of the space meets the condition L<(nπ²EI/F)^{1/2}, where n=4 or lower, E is an elastic modulus of a material of a polymeric structure spanning the space, F is an axial loading on the polymeric structure, and I is a moment of inertia of a cross section of the polymeric structure.

Optionally, a space between one of the connecting members in the first set and the second ring element has a width L, wherein the catheter further comprises a polymeric structure spanning the space, the polymeric structure having an elastic modulus E, and wherein a ratio E/L² meets the below condition: F/(nπ2I)<E/L², where F is an axial loading on the polymeric structure, n=4 or lower, and I is a moment of inertia of a cross section of the polymeric structure.

Optionally, the catheter further includes a marker element attached to or extending from a distal end of the tubular structure.

Optionally, the marker element comprises a ring structure having a distal end, a proximal end, and a body extending between the distal end and the proximal end, wherein the ring structure is made from a radiopaque material; wherein the distal end of the ring structure comprises protruding elements disposed circumferentially around an axis of the ring structure; and wherein the proximal end of the ring structure is configured to couple with, or extends from, the tubular structure.

Optionally, the protruding elements comprise respective curvilinear tip surfaces, wherein the distal end of the ring structure further comprises curvilinear trough surfaces, wherein each of the curvilinear trough surfaces is disposed between two adjacent ones of the curvilinear tip surfaces.

Optionally, the curvilinear tip surfaces and the curvilinear trough surfaces together form a sinusoidal profile extending circumferentially around the axis of the ring structure.

Optionally, the catheter further comprises a first polymer layer disposed circumferentially around an exterior surface of the tubular structure.

Optionally, the first polymer layer comprises a first segment and a second segment proximal to the first segment.

Optionally, the first segment is made from NEUSoft.

Optionally, the second segment is made from a material that is different from NEUSoft.

Optionally, the catheter further includes a second polymer layer disposed at an interior surface of the tubular structure.

Optionally, the second polymer layer comprises a first segment and a second segment proximal to the first segment, wherein the first segment of the second polymer layer is made from NEUSoft.

Optionally, the second segment of the second polymer layer is made from a material different from NEUSoft.

Optionally, a first segment of the first polymer layer and a first segment of the second polymer layer are made from a same material.

Optionally, the catheter further includes a marker element having a ring structure, and a plurality of holes disposed circumferentially at a body of the ring structure, wherein parts of the first polymer layer extend into the holes of the ring structure.

Optionally, the first polymer layer extends distally past a distal tip of the marker element to form a polymeric tip.

Optionally, the polymeric tip extends less than 3 mm beyond a distal-most tip of the marker element.

Optionally, the first polymer layer has a tensile modulus that is at least 2 Mpa and/or at most 7 Mpa, a tensile elongation (strain) at yield that is at least 300%, a tensile elongation (strain) at breakpoint that is at least 500%, or any combination of the foregoing.

Optionally, the second polymer layer has a tensile modulus that is at least 2 Mpa and/or at most 7 Mpa, a tensile elongation (strain) at yield that is at least 300%, a tensile elongation (strain) at breakpoint that is at least 500%, or any combination of the foregoing.

Optionally, the first polymer layer and the second polymer layer form a tubing encapsulating the tubular structure; and wherein a material of the tubing has a modulus elasticity that is higher than a material of the tubular structure, and is more elastic than the material of the tubular structure.

Optionally, a stiffness of a combination of the tubular structure and the tubing is at least four times a stiffness of the tubular structure alone.

Optionally, the tubular structure has a wall gap to kerf width ratio that is anywhere from 1 to 5.

Other and further aspects and features of embodiments will become apparent from the ensuing detailed description in view of the accompanying figures.

### BRIEF DESCRIPTION OF THE FIGURES

FIG. 1 illustrates a catheter in accordance with some embodiments.

FIG. 2A illustrates a distal segment of the catheter of FIG. 1, particularly showing the distal segment of the catheter having a marker device and a tubular structure.

FIG. 2B illustrates the tubular structure of FIG. 2A.

FIG. 2C illustrates a close-up view of a junction in the tubular structure of FIG. 2A

FIG. 2D illustrates a widening of a lumen of the tubular structure of FIG. 2A due to an auxetic angle of ring elements in the tubular structure.

FIG. 2E illustrates the tubular structure of FIG. 2A under tension.

FIG. 2F illustrates the tubular structure of FIG. 2A undergoing bending.

FIG. 2G illustrates a close-up view of the tubular structure of FIG. 2C, particularly showing a gap-width-control bump.

FIG. 3 illustrates the marker device of FIG. 2A.

FIGS. 4-5 illustrate a variation of the marker device of FIG. 3, particularly showing the marker device having multiple tabs.

FIG. 6 illustrates a variation of the marker device of FIG. 4, particularly showing the marker device having multiple openings.

FIG. 7 illustrates a distal segment of the catheter of FIG. 1.

FIG. 8 illustrates an example of the catheter of FIG. 1, particularly showing the catheter having a first layer outside the tubular structure, and a second layer inside the tubular structure.

FIGS. 9A-9B illustrate another tubular structure that is different from the tubular structure of FIG. 2B.

### DETAILED DESCRIPTION

Various embodiments are described hereinafter with reference to the figures. It should be noted that elements of similar structures or functions are represented by the same reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure. In some cases, the term “about” may refer to a range of values that are within +/−10% of a value. For example, a value of 2 or a value of about 2 may refer to any value that is within the range of 2+/−10% (=2+/−0.2=1.8 to 2.2).

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

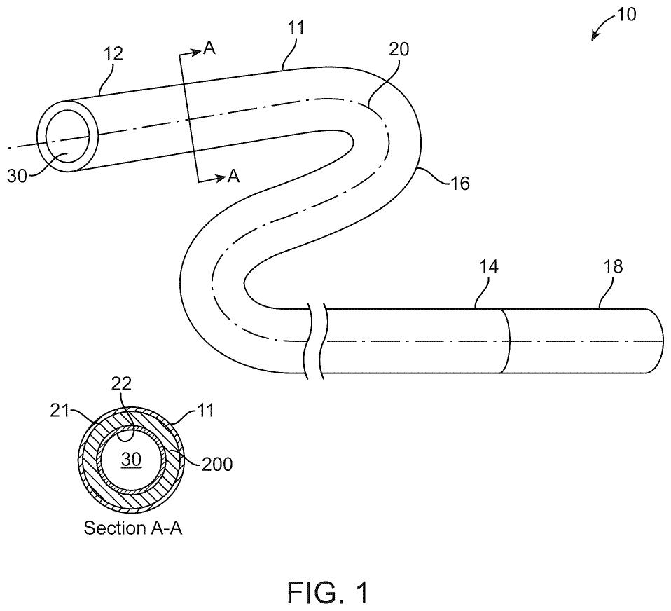

FIG. 1 illustrates a catheter 10 in accordance with some embodiments. The catheter 10 includes a tube 11 having a distal end 12, a proximal end 14, and a tube body 16 extending between the distal end 12 and the proximal end 14. The catheter also includes a handle 18 attached to the proximal end 14 of the tube 11.

The tube 11 includes an outer surface 21, and inner surface 22, and a lumen defined by the inner surface. The tube 11 also includes a tubular structure 200 configured to provide certain stiffness for the tube 11. As shown in the figure, the tubular structure 200 is disposed between the outer surface 21 and the inner surface 22 of the tube 11, so that the tubular structure 200 is embedded within a wall of the tube 11. In other embodiments, the tubular structure 200 may be on the outer surface 21, or on the inner surface 22 of the tube 11. The tubular structure 200 has a distal end, a proximal end, and a body extending between the distal end and the proximal end. The tubular structure 200/catheter 10 also has a longitudinal axis 20 defined by the distal end and the proximal end of the tubular structure 200.

In the illustrated embodiments, the lumen 30 of the catheter 10 has a cross-sectional shape when the catheter 10 is in a relaxed state. The tubular structure 200 is configured to maintain the cross-sectional shape of the lumen 30 during bending of the catheter 10, so that the catheter 10 will not kink. Optionally, the tubular structure 200 may also be configured to provide axial stiffness, bending stiffness, torsional stiffness, or any combination of the foregoing, for the catheter 10.

### Tubular Structure

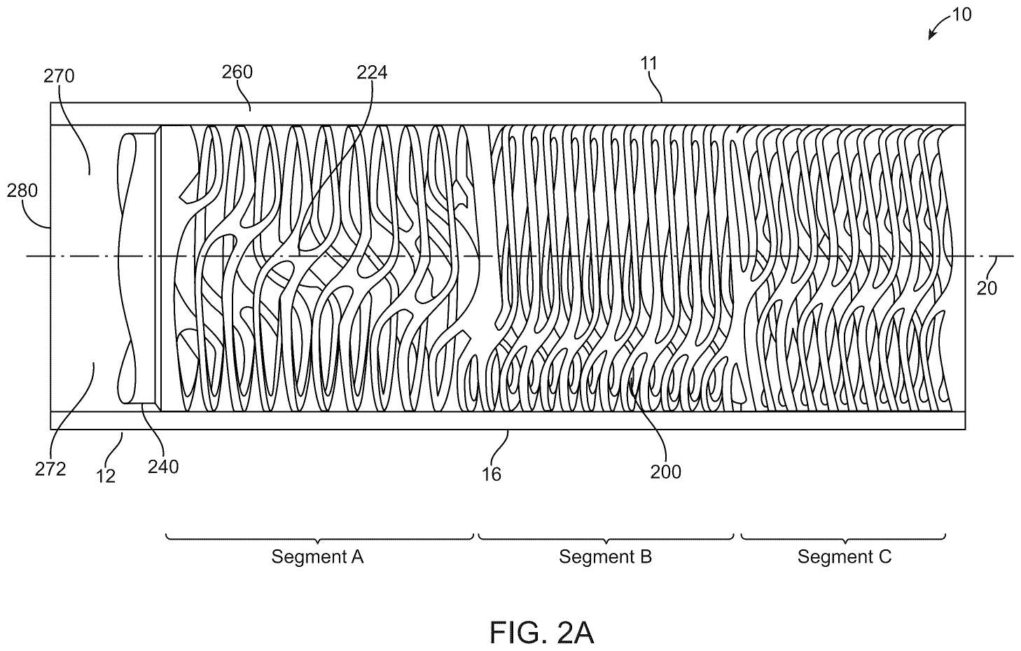

FIG. 2A illustrates a distal segment of the catheter 10 of FIG. 1, particularly showing the distal segment of the catheter 10 having a marker element (marker device) 240 and a tubular structure 200. The tubular structure 200 is configured to provide axial stiffness, bending stiffness, torsional stiffness, or any combination of the foregoing, for the catheter 10. In some cases, the tubular structure 200 may comprise a hypotube.

The distal segment of the catheter 10 also includes a polymer layer 260 disposed circumferentially around the marker element 240 and/or the tubular structure 200. The distal segment of the catheter 10 further includes a polymeric tip 270. The polymeric tip 270 comprises a polymeric tube 272 having a distal tip 280. The polymeric tip 270 is distal to the marker element 240 and/or the tubular structure 200. In some cases, the polymeric tip 270 may be integral with the polymer layer 260. For example, the polymeric tip 270 may be formed together with at least a part of the polymer layer 260 using a same material. In other cases, the polymeric tip 270 and the polymer layer 260 may be formed separately, and are then connected together, e.g., using adhesive, fusing technique, etc. In such cases, the polymeric tip 270 and the polymer layer 260 may be made from the same material, or different respective materials.

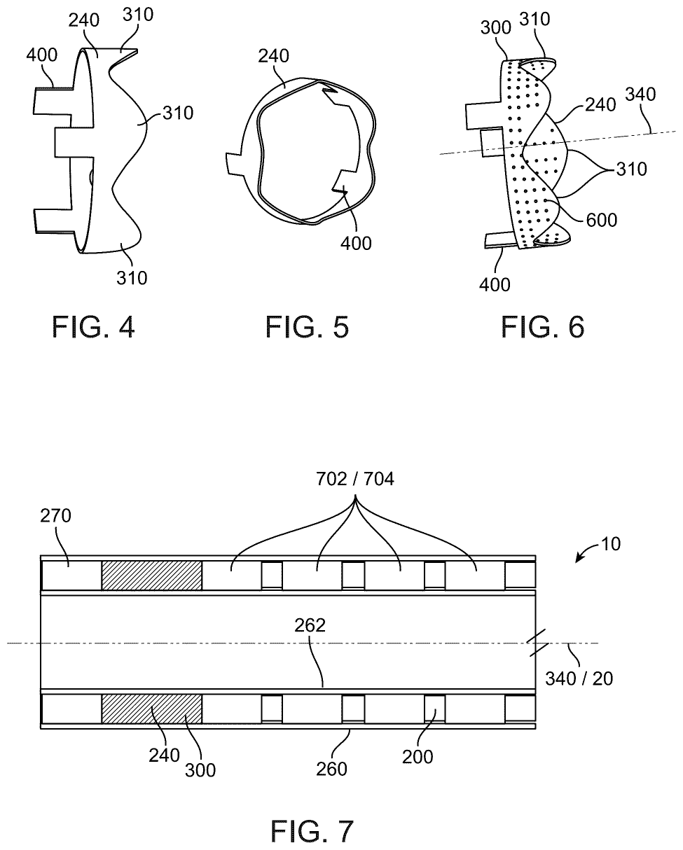

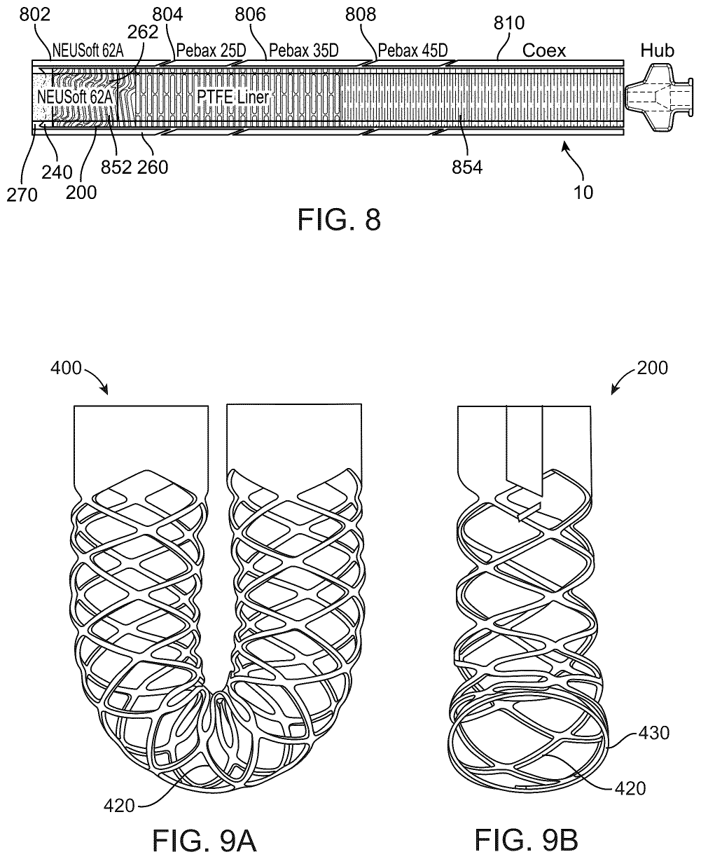

Also, the distal segment of the catheter 10 may also include a polymer layer 262 (shown in FIG. 8) disposed at the inner surface of the marker element 240 and/or at the inner surface of the tubular structure 200. In some cases, the polymeric tip 270 may be integral with the polymer layer 262. For example, the polymeric tip 270 may be formed together with at least a part of the polymer layer 262 using a same material. In other cases, the polymeric tip 270 and the polymer layer 262 may be formed separately, and are then connected together, e.g., using adhesive, fusing technique, etc. In such cases, the polymeric tip 270 and the polymer layer 262 may be made from the same material, or different respective materials. The polymer layers 260, 262 will be further described with reference to FIGS. 8-9.

FIG. 2B illustrates the tubular structure 200 of FIG. 2A. The tubular structure 200 is configured to provide axial stiffness, bending stiffness, torsional stiffness, or any combination of the foregoing, for the catheter 10.

The tubular structure 200 has a plurality of ring elements 210 arranged in series along the longitudinal axis 20. In the illustrated embodiments, the ring elements 210 are respective closed-loops. In the figure, three of the ring elements 210 are identified (e.g., a first ring element 210a, a second ring element 210b, and a third ring element 210c). The tubular structure 200 may include any number of ring elements 210. As shown in the figure, each ring element 210 has multiple ring portions 222 and multiple joint portions 224. Each joint portion 224 is between two adjacent ring portions 222. The ring portions 222 and the joint portions 224 are connected circumferentially to form the ring element 210.

Each ring portion 222 of the ring element 210 is a partial ring, and a part of each ring portion 222 has a rectilinear configuration when viewed from a side like that shown in FIG. 2B. The rectilinear part (or flat part) of the ring portion 222 lies in a plane that forms an acute angle 286 with respect to the longitudinal axis 20 (e.g., when viewed from a side of the tubular structure like that shown in the figure). The acute angle may be anywhere from 60 degrees to 89 degrees, and more preferably anywhere from 75 degrees to 86 degrees. In one implementation, the acute angle may be 84 degrees +/−2 degrees. In other cases, the ring portion 222 may not include any flat part. For example, in other cases, the entire ring portion 222 may have a curvilinear configuration when viewed from a side of the tubular structure 200.

As shown in the figure, each of the first and second ring portions 222a, 222b in the ring element 210b extends in a non-perpendicular direction with respect to a longitudinal axis of the tubular structure to thereby provide a non-planar configuration for the ring element 210b. A ring element 210 is considered as having a “non-planar configuration” if different circumferential parts of the ring element 210 collectively form a surface that is not a rectilinear surface. Thus, a ring element 210 may be relatively “flat” in the sense that the entire ring element 210 may fit within a thin cylinder, the ring element 210 may still be considered as having a non-planar configuration if different circumferential parts of the ring element 210 do not collectively form a rectilinear surface. This non-planar configuration of the ring element 210 is advantageous because it allows connecting members 220 between the ring elements 210 to be packed closely next to the adjacent ring elements 210. As shown in the figure, a majority of each connecting member 220 forms a uniform spacing with a part of a first adjacent ring element 210, and also forms a uniform spacing with a part of a second adjacent ring element 210 (with the first and second adjacent ring elements 210 being on opposite sides of the connecting member 220). When the tubular structure 200 is covered or encapsulated by one or more polymeric layers forming a polymeric tube attached to the tubular structure 200, the uniform spacing described above will allow a uniform width of the polymeric material of the polymeric tube to form in the spacing. As a result, the polymeric tube attached to the tubular structure 200 will have an uniform mechanical property across the ring elements 210, and/or will not have any significant stress concentration across the ring elements during use. The connecting members 220 and the polymeric tube will be described in further detail below.

In other embodiments, the non-planar configuration is not required for each ring element 210, and each ring element 210 may instead have a planar configuration in which an entirety of the ring element 210 lies within a plane.

In the illustrated example, each ring element 210 has multiple ring portions 222 that are disposed circumferentially, so that each ring portion 222 is a circumferential part of the ring element 210. In the ring element 210, the ring portions 222 have the same configuration (e.g., same dimensions, shape, profile, etc.), such that the configuration of the ring portion 222 in the ring element 210 repeats circumferentially. The ring portions 222 of each ring element 210 do not lie in a same plane. In the cases in which each ring portion 222 has a flat (rectilinear) part, the flat parts of the respective ring portions 222 of the ring element 210 lie in different respective planes (e.g., the planes may form acute angles with respect to each other). In other cases in which each ring portion 222 has a non-flat profile (e.g., curvilinear profile, a zig-zag profile, etc.), the non-flat profile of each ring portion 222 repeats circumferentially around the ring element 210. Because the ring portions 222 in each ring element 210 have the same non-flat profile, corresponding parts of the ring portions 222 are in the same longitudinal position with respect to the longitudinal axis 20, but different parts of a ring portion 222 have different respective longitudinal positions with respect to the longitudinal axis 20.

In some cases, each ring element 210 has only two ring portions 222. In such cases, the first ring portion extends at least 40% (e.g., 50%) of a circumference of the ring element 210, and the second ring portion 222 also extends at least 40% of the circumference of the second ring element 210. In other cases, each ring element 210 may have more than two ring portions 222. For example, each ring element 210 may have three ring portions 222. In such cases, each of the first, second, and third ring portions 222 may extend at least 30% (e.g., 33%) of a circumference of the ring element 210. In further cases, each ring element 210 may have four ring portions 222. In such cases, each of the ring portions 222 may extend at least 22% (e.g., 25%) of a circumference of the ring element 210. In further cases, each ring element 210 may have five ring portions 222. In such cases, each of the ring portions 222 may extend at least 18% (e.g., 20%) of a circumference of the ring element 210.

Each joint portion 224 of a ring element 210 is connected between two adjacent ring portions 222 of the ring element 210. The joint portion 224 provides a “step” between the two adjacent ring portions 222. This allows the ring portions 222a, 222b of the ring element 210b to be connected to each other even though the ring portions 222a, 222b do not completely lie in a common plane.

In the illustrated example, each ring element 210 has two joint portions 224. In other examples, each ring element 210 may have more than two joint portions 224 (e.g., three, four, five, six, seven, eight, etc., joint portions 224).

As shown in FIGS. 2B-2C, when viewed from a side, the joint portion 224 of the ring element 210 has a central portion 225, a first curved portion 226, and a second curved portion 227. In some cases, the central portion 225 of the joint portion 224 is configured to rotate and/or to resist a moment when the catheter 10 is being tensioned or is undergoing bending. The first curved portion 226 and the second curved portion 227 are on opposite sides of the central portion 225. The first curved portion 226 and the second curved portion 227 are connected to respective ring portions 222 on opposite sides of the central portion 225. As shown in the figure, the amount of curvature is the same for both the first curved portion 226 and the second curved portion 227, but the direction of curvature of the first curved portion 226 is different from the direction of curvature of the second curved portion 227 (e.g., they are curving at opposite directions). In other cases, the amount of curvature of the first curved portion 226 may be different from the amount of curvature of the second curved portion 227. Also, in other cases, instead of having the curvilinear configuration, the first portion 226 and the second portion 227 may each have a rectilinear configuration. For example, in other cases, the first portion 226, the second portion 227, and the central portion 225 may have respective rectilinear configurations (e.g., when viewed from a side) forming a zig-zig shape.

Furthermore, in other cases, the portions 226, 227 may be considered parts of the respective ring portions 222 in the ring element 210. In such cases, the joint portion 224 includes the central portion 225, but not the portions 226, 227.

The tubular structure 200 also includes connecting members 220 connected between adjacent ones of the ring elements 210. During use, the ring elements 210 provides structural stiffness to maintain a shape of the central lumen of the tubular structure 200 while the catheter 10 is being bent. At the same time, the connecting members 220 maintain the structural integrity of the tubular structure 200 by allowing the spacing between adjacent ring elements 210 to vary predictably as the catheter is being bent. The connecting members 220 also provides bending stiffness, axial stiffness, torsional stiffness, or any combination of the foregoing for the tubular structure 200. Thus, the ring elements 210 and the connecting members 220 are two different groups of structural components for the tubular structure 200 that behave differently during use.

As shown in FIG. 2B, the tubular structure 200 includes a first set of connecting members 220a connected between the first ring element 210a and the second ring element 210b. The tubular structure 200 also includes a second set of connecting members 220b connected between the second ring element 210b and the third ring element 210c. Each connecting member 220a/220b includes a first member end 232, a second member end 234 opposite from the first member end 232, and a member body 236 extending between the first member end 232 and the second member end 234. The member body 236 may form an angle with respect to the adjacent ring elements 210 to which the member body 236 is attached. In the illustrated example, the angle formed between the member body 236 and the ring element 210 (e.g., the ring portion 222 of the ring element 210) is 0 degree when the tubular structure 200 is in a relaxed configuration. This means that when the tubular structure 200 is in the relaxed configuration, the member body 236 of the connecting member 220 is parallel to a part (e.g., the ring portion 222) of the ring element 210. In other cases, the angle may be an acute angle that is larger than 0 degree, but less than 90 degrees. In some cases, the angle may be measured with the tubular structure 200 being “un-rolled” to a flat configuration.

In some cases, the tubular structure 200 may have a total of two connecting members 220 between each adjacent pair of ring elements 210. In other examples, the tubular structure 200 may have more than two (e.g., three, four five, six, seven, eight, etc.) connecting members 220 between each adjacent pair of ring elements 210.

Also, the first member end 232 of each connecting member 220 and the second member end 234 of each connecting member 220 may define a line that is non-parallel to the longitudinal axis 20 (e.g., when viewed from a side of the tubular structure 200). As shown in FIG. 2B, the member body 236 of the connecting member 220 forms an angle 288 with respect to the longitudinal axis 20. The angle 288 may be any angle that is larger than 0 degree, and less than 90 degrees. By means of non-limiting examples, the angle 288 may be larger than 70 degrees, larger than 80 degrees, larger than 85 degrees, etc.

In the illustrated examples, the member body 236 is a major part of each connecting member 220. Also, the member end 232/234 of the connecting member 220 is a minor part that connects to a ring element 210. In some cases, the member body 236 may constitute at least 70%, or at least 80%, or at least 90%, or at least 95% of an entire length of the connecting member 220. In such cases, the member end 232/234 may constitute the remaining percentage of the length of the connecting member 220. The member end 232/234 is illustrated as having a curvilinear configuration. In other cases, the member end 232/234 may have a rectilinear configuration.

Also, in some cases, the member body 236 may constitute 100% of the entire length of the connecting member 220. In such cases, the member end 232/234 will constitute an end point at an end of the member body 236, and will not have any dimension.

FIG. 2C illustrates a junction 229 in which two connecting members 220 are connected to a ring element 210. As shown in the figure, the junction 229 is formed by an end 234 of a connecting member 220, an end 232 of another connecting member 232, the central portion 225 of the joint portion 224 of the ring element 210, the first portion 226 of the joint portion 224 of the ring element 210, and the second portion 227 of the joint portion 224 of the ring element 210.

Also, as shown in FIG. 2C, each ring element 210 is slanted with respect to the longitudinal axis 20, and forms an “auxetic angle” with respect to a plane that is perpendicular to the longitudinal axis 20. The auxetic angle is advantageous because it allows the ring element 210 to maintain, or even “enlarge”, its central lumen while the tubular structure 200 is undergoing bending and/or tension. During use of the catheter 10, in response to bending or tension applied to the tubular structure 200, the ring element 210 of the tubular structure 200 may rotate about an axis that is perpendicular to the longitudinal axis 20. As a result, as shown in FIG. 2D, the ring element 210 may move from a first position P1 in which the ring element 210 is at a slanted angle with respect to the longitudinal axis 20 (or at an auxetic angle A with respect to a plane perpendicular to the longitudinal axis 20), to a second position P2 in which the ring element 210 is perpendicular to the longitudinal axis 20. This, in turn, results in a “widening” of the central lumen of the tubular structure 200. Note that in FIG. 2D, the connecting members 220 are not shown for clarity. As shown in the figure, when the ring element 210 is at the first position P1, the lumen of the tubular structure 200 has a first width W1. However, as the tubular structure 200 is stressed, the ring element 210 moves from the first position P1 to the second position P2, resulting in a widening of the lumen from the first width W1 to a second width W2. Accordingly, the auxetic angle of the ring element 210 effectively provides a positive poisson's ratio for the tubular structure 200 in the sense that as the tubular structure 200 is tensioned, the cross-sectional dimension of the tubular structure 200 increases. In other words, as the tubular structure 200 is being tensioned in a direction that is parallel to the longitudinal axis 20, the lateral dimension of the tubular structure 200 increases (due to the ring element 210 being rotated from a slanted orientation to an orientation in which the ring element 210 is perpendicular to the longitudinal axis 20). This is contrary to the behavior of many materials that have a negative poisson's ratio, which is defined as the negative ratio of transverse strain to axial strain. For any of these materials, when tension is applied to the material, the material will elongate in the direction of tension, and will also decrease in dimension in the lateral direction (that is perpendicular to the direction of tension).

In some cases, the number N of connecting members 220 between two ring elements 210 may be based on (1) an axial load demand F (expected maximum axial force, such as compression or tension) on the catheter 10, (2) an area A of a cross section of each connecting member 220, (3) allowable axial stress S for each connecting member 220, or (4) any combination of the foregoing. In one implementation, N≥F/(S*A). Also, the area A of the cross section of the connecting member 220 may be thickness t times a width w of the connecting member 220 in the situation in which the cross section of the connecting member 220 is a rectangle, or in the situation in which the cross section is not a rectangle but can be approximated as a rectangle. In some cases, the thickness t of the connecting member 220 may be the wall thickness of the tubular structure 200, or a dimension that is measured in a radial direction with respect to the cross section of the connecting member 220. Also, in some cases, the width w of the connecting member 220 may be a dimension that is measured in a tangential or circumferential direction with respect to the cross section of the connecting member 220. By means of non-limiting examples, the axial load demand F for the catheter 10 may be 2 Newtons or higher, 3 Newtons or higher, 4 Newtons or higher, 5 Newtons or higher, etc.

Also, to ensure that each connecting member 220 will move relative to adjacent ring elements 210 in a tangential direction (i.e., the direction that is perpendicular to a radial direction with respect to the cross section of the tubular structure 200), instead of moving in a radial direction with respect to the cross section of the tubular structure 200, the thickness t of the connecting member 220 is designed to be larger than the width w of the connecting member 220 (such that t>w).

In some cases, to help meets the condition t>w for each connecting member 220, a splitting technique may be used for the design of the connecting members 220. For example, if an initial design of a connecting member 220 has a width w that is larger than its thickness t, then the initial designed connecting member may be “split” into two halves, so that the width w of the initial design is reduced by 50%. In other cases, the splitting may be performed to split into more than two parts (e.g., three parts, four parts, etc.) so that the condition t>w is met for each connecting member 220.

In some cases, the tubular structure 200 is designed so that t>w for each connecting member 220, and so that N*t*w*S>F. In one design technique, these two requirements may be considered together. For example, for a given number N of connecting members 220, the above two equations may be utilized to determine t and w. Alternatively, for a given t and a given w, the minimum number N of connecting members 220 may be determined.

FIG. 2E illustrates the tubular structure 200 in tension. The tubular structure 200 of FIG. 2E is the same as the segment A of the tubular structure 200 of FIG. 2A, except that it has three connecting members 220 (instead of two connecting members 220) between two adjacent ring elements 210. As shown in the figure, the member body 236 of each connecting member 220 rotates and/or bends relative to the adjacent ring element 210 as the tubular structure 200 is being axially loaded (e.g., tensioned). Accordingly, the angle 288 changes as the tubular structure 200 is being axially loaded. In addition, as the tubular structure 200 is being axially loaded to change (e.g., to increase or decrease) a spacing between adjacent ring elements 210, the cross-sectional shape of the ring elements 210 are maintained, and the angle between the ring elements 210 and the longitudinal axis 20 is also maintained.

FIG. 2F illustrates the tubular structure 200 undergoing bending. The tubular structure 200 of FIG. 2E is the same as the segment A of the tubular structure 200 of FIG. 2A, except that it has three connecting members 220 (instead of two connecting members 220) between two adjacent ring elements 210. As shown in the figure, when the tubular structure 200 is being bent, the angle between the ring elements 210 and the longitudinal axis 20 stay substantially the same (e.g., the angle does not change by more than 10% compared to when the tubular structure 200 is not in bending). The connecting members 220 are configured to move relative to the ring elements 210 in correspondence with the bending of the tubular structure 200. This allows the connecting members 220 to conform to a change in a spacing distance between adjacent ring elements 210 due to the bending of the tubular structure 200.

The tubular structure 200 is advantageous because the close-loop ring elements 210 prevents the tubular structure 200 from collapsing radially inward. Thus, the cross-sectional shape of the ring elements 210 are maintained even during bending of the tubular structure 200, and kinking of the catheter 10 is prevented.

As shown in FIG. 2C, the tubular structure 200 also includes gap-width-control bumps 238. Each gap-width-control bump 238 is located along a side of the ring element 210, and is configured to ensure that a gap width between the ring element 210 and adjacent connecting member 220 does not exceed a prescribed gap limit. The gap-width-control bump 238 is illustrated in the close-up view of FIG. 2G. As shown in the figure, a prescribed gap width limit 239a for the tubular structure 200 is shown. If the gap-width-control bump 238 is not provided, the side of the ring element 210 will have the profile shown in dashed-line, and the gap width 239b between such portion of the ring element 210 and the adjacent connecting member 220 will exceed the dimension of the prescribed gap width limit 239a. The introduction of the gap-width-control bump 238 reduces such gap width 239b to gap width 239c that is less than or equal to the prescribed gap width limit 239a. The gap-width-control bumps 238 are advantageous because they prevent gaps between elements of the tubular structure 200 to exceed a threshold, thereby controlling a size of a polymer accommodated between elements of the tubular structure 200. This in turn may reduce the risk of structural failure at the interface between the polymer and the element of tubular structure 200. In other embodiments, the gap-width-control bumps 238 may be implemented along sides of respective connecting members 220, instead of being along sides of the ring elements 210. In further embodiments, the gap-width-control bumps 28 may be implemented along sides of respective connecting members 220 and along sides or respective ring elements 210.

FIGS. 9A-9B illustrate a bending of another tubular structure 900 that is different from the tubular structure 200 of FIG. 2A. The tubular structure 900 comprises members 420 that are arranged in crisscross configuration. Unlike the tubular structure 200, the tubular structure 900 does not have any closed-loop ring elements that lie in respective planes. As shown in FIG. 9B, during bending of the tubular structure 900, the tubular structure 900 may be “squished” due to one side of the tubular structure 900 being in compression from the bending. As a result, if the tubular structure 900 defines a circular cross-section lumen while in a relaxed state, the “squished” tubular structure 900 may result in the lumen having an elliptical shape 430. A catheter constructed using the tubular structure 900 will be easily kinked during use.

As discussed, the connecting members 220 (i.e., a first set of connecting members 220, and a second set of connecting members 220) on both sides of the ring element 210 are configured to move relative to the ring element 210 during bending and/or axial loading of the catheter 10. In some cases, as the tubular structure 200 is being tensioned, the angle between each ring element 210 and the longitudinal axis stay substantially the same (e.g., the angle does not vary by more than 10% compared to when the tubular structure 200 is not in tension). The connecting members 220 between adjacent pairs of the ring elements 210 elastically flex in correspondence with axial movement of the ring elements 210 as the tubular structure 200 is being tensioned. Also, the member body 236 of each connecting member 220 rotates and/or bends relative to the adjacent ring element 210 so that the angle 288 changes as the tubular structure 200 is being tensioned. In addition, as the tubular structure 200 is being tensioned to change a spacing between adjacent ring elements 210, the cross-sectional shape of the ring elements 210 are maintained.

Also in some cases, the tubular structure 200 can undergo very tight bending to have a bent shape with small radius of curvature. Through the entire length of the bent, the gaps between respective pairs of adjacent ring elements 210 remain substantially even (e.g., the gaps between different pairs of adjacent ring elements 210 do not vary by more than 30%, or more preferably do not vary by more than 20%, or more preferably do not vary by more than 10%). This is the case for both the gaps on the tension side, and the gaps on the compression side, of the tubular structure 200. In other cases, the gaps between different pairs of adjacent ring elements 210 may vary by more than 30% during bending of the catheter 10.

In some cases, while the catheter 10 is being tensioned, the gaps between respective pairs of adjacent ring elements 210 remain substantially even (e.g., the gaps between different pairs of adjacent ring elements 210 do not vary by more than 30%, or more preferably, do not vary by more than 20%, or more preferably, do not vary by more than 10%). In other cases, the gaps between different pairs of adjacent ring elements 210 may vary by more than 30% during tensioning of the catheter 10.

Also, in some cases, the tubular structure 200 can undergo 360-degree bending to have a bent shape with small radius of curvature. Through the entire length of the bent, the gaps between respective pairs of adjacent ring elements 210 remain substantially even (e.g., the gaps between different pairs of adjacent ring elements 210 do not vary by more than 30%, or more preferably, od not vary by more than 20%, or more preferably, do not vary by more than 10%). This is the case for both the gaps on the tension side, and the gaps on the compression side, of the bent tubular structure 200. In some cases, even with such extreme bending, the tubular structure 200 forms no kink, and the structural integrity of the tubular structure 200 is maintained. In other cases, the gaps between different pairs of adjacent ring elements 210 may vary by more than 30% during 360-degree bending of the tubular structure 200.

As shown in FIG. 2A, the tubular structure 200 has multiple segments A-C aligned longitudinally. The segments A-C have different respective cut-patterns, which provide different respective stiffnesses (or flexibilities) for the segments A-C. In some cases, each of the segments may be implemented using a tube segment with cutouts, and the tube segments may be coupled together along the longitudinal axis. In other cases, two or more segments of the tubular structure 200 may be implemented with a tube segment. In further cases, all of the segments implementing the entirety of the tubular structure 200 may be implemented using a single tube segment.

In some cases, a first segment of the tubular structure 200 may be more flexible (e.g., having more bending flexibility, axial flexibility, torsional flexibility, or any combination of the foregoing) than a second segment of the tubular structure 200, wherein the second segment is proximal to the first segment. Also, in some cases, the second segment may be more flexible than a third segment of the tubular structure 200, wherein the third segment is proximal to the second segment. In some cases, the bending stiffness of the tubular structure 200 reduces from a proximal end of the tubular structure 200 to a distal end of the tubular structure 200.

In the illustrated example shown in FIG. 2A, the segments A-C have the same configurations (e.g., they all have ring elements 210 with ring portions 222 and joint portions 224, and connecting members 220), except that the numbers of joint portions 224 and the numbers of connecting members 220 between adjacent ring elements 210 are different among the segments A-C. In particular, in segment A of the tubular structure 200, each ring element 210 has a total of two joint portions 224 and two ring portions 222, with the two joint portions 224 connecting to two respective connecting members 220. In segment B of the tubular structure 200, each ring element 210 has a total of three joint portions 224 and three ring portions 222, with the three joint portions 224 connecting to three respective connecting members 220. In segment C of the tubular structure 200, each ring element 210 has a total of four joint portions 224 and four ring portions 222, with the four joint portions 224 connecting to four respective connecting members 220. Since increasing the number of connecting members 220 between adjacent ring elements 210 will have the effect of stiffening the segment of the tubular structure 200, the above configurations result in a tubular structure 200 having a first segment (segment A) that is the most flexible, a second segment (segment B-proximal to the first segment) that is stiffer than the first segment, and a third segment (segment C-proximal to the second segment) that is stiffer than the second segment.

It should be noted that the tubular structure 200 is not limited to having all of the segments A-C. In other cases, the tubular structure 200 may have fewer than three segments (e.g., one segment or two segments), or more than three segments (e.g., four, five, six, seven, eight, nine, ten segments). Also, in other cases, the tubular structure 200 may have a segment A with the configuration shown in the figure, but it may also have segments B, C with configurations that are different from that shown in the figure.

As shown in FIG. 2B, the tubular structure 200 includes space 216 defined between the ring elements 210 and the connecting members 220. In some embodiments, filler may be disposed in the space 216 defined between the ring elements 210 and the connecting members 220. The filler provides a seal to prevent fluid from passing across a wall of the tubular structure 200. The filler may be a material extending from the outer polymer layer 260, a material extending from the inner polymer layer 262, a material separate from the polymer layers 260, 262, or any combination of the foregoing. In the illustrated example, because the connecting members 220 are parallel to the ring portions 222 of the ring elements 210, the space 216 between them has a uniform width. This allows the filler to have a same corresponding width that is uniform (constant) when filling the space 216. This feature is advantageous because it reduces or prevents stress concentration at the filler, reduces the risk of the filler failing during use, reduces the risk of the filler being separated from the tubular structure 200, and allows the filler to couple and work better with the components of the tubular structure 200.

It should be noted that the tubular structure 200 may be any member having a tube configuration, and is not limited to the example of the configuration shown in FIGS. 2A-2E. For examples, in other cases, the ring elements 210 and/or connecting member 220 may have variations in length, width and thickness to adjust flexibility and kink resistance of the tubular structure 200.

In any of the embodiments of the tubular structure 200 described herein, the tubular structure 200 may be made from a raw tube. The raw tube may be a metal tube, an alloy tube, a plastic tube, a polymeric tube, or a tube made from any of other materials. The raw tube is then cut to form the ring elements 210 and connecting members 220. In such cases, the ring elements 210 and the connecting members 220 are parts of a cut tube. The cutting of the raw tube may be performed using laser cutting in some embodiments. For example, an electronic file storing geometric information (e.g., shape information, dimension information, etc.) regard the tubular structure 200 to be formed may be created. The electronic file may be provided to a processing unit of a laser cutting machine. The processing unit processes the electronic file to operate a laser cutter of the laser cutting machine to cut geometric pattern(s) defined by the information in the electronic file. In some embodiments, the laser cutting may be performed on the raw tube. In other cases, instead of a raw tube, a raw sheet of material may be provided, and the laser cutting may be performed on the raw sheet. After the laser cutting is performed, the cut sheet may be rolled to form the tubular structure 200. Edges (that are parallel to the longitudinal axis) of the rolled sheet may be connected together to form a closed loop tube. Other techniques involve forming the desired pattern into a sheet or a tube by chemical etching or electrical discharge machining.

In general, the tubular structure 200 may be made from a “removal” process in which some materials are removed from a raw tube to create openings (e.g., elongated slots) through a wall of the tube. The material removal may be accomplished by laser-cutting, etching, mechanical cutting, sanding, polishing, etc. In any of these techniques, the created openings may be considered “cutouts” that provide a certain mechanical property (e.g., flexibility) for the catheter 10.

In other cases, the ring elements 210 and the connecting members 220 may be integrally formed together. For example, a mold having a rod with protrusions on the surface of the rod may be provided. The protrusions correspond with the space 216 to be formed between the ring elements 210 and the connecting members 220. Then the material for forming the ring elements 210 and the connecting members 220 is deposited onto the mold. The material is then cured to form the ring element 210 and the connecting members 220.

In further cases, the ring elements 210 and the connecting members 220 may be separately formed, and are then connected to each other after they are formed. In one implementation, a plurality of ring elements 210 may be provided. Then a tubular mesh may be used to connect the ring elements 210 in series. In particular, the ring elements 210 in spaced-away configuration may be disposed over the tubular mesh, and are arranged in series along a longitudinal axis of the tubular mesh. The ring elements 210 may then be secured to the tubular mesh, such as via an adhesive, glue, weld, etc. Parts of the tubular mesh between the ring elements 210 become and function as the connecting members 220.

As discussed, in some embodiments, the catheter 10 may include an outer layer 260 (e.g., an outer sheath) disposed circumferentially around the tubular structure 200, and/or an inner layer 262 (e.g., an inner sheath) disposed at an inner surface of the tubular structure 200. If the catheter 10 includes both the outer layer 260 and the inner layer 262, the tubular structure 200 is sandwiched there-between.

In some cases, material of the outer layer 260 and/or material of the inner layer 262 may fill the space 216 defined by elements of the wall of the tubular structure 200. In such cases, the material from the outer layer 260 and/or the material from the inner layer 262 may form a filler (e.g., the filler 250) that fills the space at the wall of the tubular structure 200.

For example, a filler may be disposed in the space (e.g., gaps) 216 between the ring elements 210 and the connecting members 220. In some embodiments, the tubular structure 200 may be dipped into a polymeric solution to allow the polymeric solution to fill the space 216. The polymeric solution may then be cured to form the filler. Excess filler material may be removed using agent, by cutting, sanding, etc.

Also, in some cases, the filler occupying the space 216 may have the same thickness as the thickness of the wall of the tubular structure 200. In other cases, the filler may be thicker than the thickness of the wall of the tubular structure 200. In further cases, the filler in the space 216 may be thinner than the thickness of the wall of the tubular structure 200.

In addition, in some cases, the material of the filler may extend pass the exterior surface of the tubular structure 200. In some cases, the material of the filler may be disposed on the exterior surface of the tubular structure 200 to form an outer layer (e.g., layer 260) covering the tubular structure 200. The outer layer may be formed integrally together with the filler in the space 216. In other embodiments, the outer layer may be formed separately from the filler, and is disposed on the exterior surface of the tubular structure 200 after the filler is disposed (e.g., formed) in the space 216. In such cases, the outer layer may be made from the same material as the filler, or may be made from a material that is different from the filler.

Similarly, in some embodiments, the material of the filler may extend pass the inner surface of the tubular structure 200. In some cases, the material of the filler may be disposed on the inner surface of the tubular structure 200 to form an inner layer (e.g., 260) covering the inner wall of the tubular structure 200. The inner layer may be formed integrally together with the filler in the space 216. In other embodiments, the inner layer may be formed separately from the filler, and is disposed on the inner surface of the tubular structure 200 after the filler is disposed (e.g., formed) in the space 216. In such cases, the inner layer may be made from the same material as the filler, or may be made from a material that is different from the filler.

In addition, in some cases, the ring elements 210 may have the same thickness as the connecting members 220. This may be the case when the ring elements 210 and the connecting members 220 are formed from a same tube or same sheet. In other cases, the ring elements 210 and the connecting members 220 may have different thicknesses. For example, in other cases, the ring elements 210 may have a first thickness, and the connecting members 220 may have a second thickness, wherein the first thickness may be larger or smaller than the second thickness.

### Marker Element

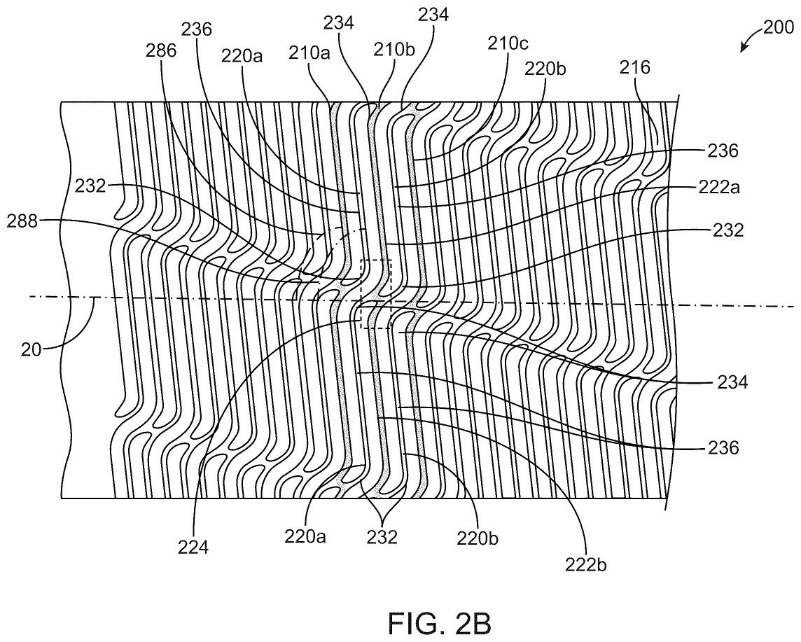

FIG. 3 illustrates the marker element 240 of FIG. 2A. As shown in the figure, the marker element 240 includes: a ring structure 300 having a distal end 301, a proximal end 302, and a body 304 extending between the distal end 301 and the proximal end 302. The ring structure 300 may be made from a radiopaque material, or any material that may allow visualization of the marker device 300 using any imaging technique (e.g., x-ray, CT, MRI, ultrasound, camera, etc.). The distal end 301 of the ring structure 300 comprises protruding elements 310 disposed circumferentially around an axis 340 of the ring structure 300. The proximal end 302 of the ring structure 300 is configured to couple with, or extends from, the tubular structure 200.

As shown in FIG. 3, the protruding elements 310 comprise respective curvilinear tip surfaces 320. The distal end 301 of the ring structure 300 further includes curvilinear trough surfaces 330. As shown in the figure, each of the curvilinear trough surfaces 330 is disposed between two adjacent ones of the curvilinear tip surfaces 320. In some cases, the curvilinear tip surfaces 320 and the curvilinear trough surfaces 330 together form a sinusoidal profile extending circumferentially around the axis 340 of the ring structure 300. In some cases, the number of the tip surfaces 320/protruding elements 310 may be two, three, four, five, six, seven, or eight. In other cases, the number of tip surfaces 320/protruding elements 310 may be more than eight.

In other embodiments, each protruding elements 310 may have a shape that is different from the example shown. For example, in other embodiments, each protruding element 310 may have a rectangular shape, a partial circular shape, an elongated shape, etc. Also, in the illustrated embodiments, each protruding element 310 has a length and a width, wherein the length is measured parallel to the longitudinal axis 340 and is shorter than the width of the protruding element 310. In other embodiments, each protruding element 310 may have a length and a width, wherein the length is measured parallel to the longitudinal axis 340 and is longer than the width of the protruding element 310.

As illustrated in the above examples, the protruding elements 310 have respective distal tips (having respective edges or tip surfaces 320) facing distally. In some embodiments, the distal end of the ring structure 300 may have a laser-cut edge.

The marker element 240 is advantageous because it is more flexible compared to another marker device having the same longitudinal length and thickness as those of the marker element 240, and being made from the same material as that of the marker element 240. By not having some materials in an alternating pattern circumferentially around the marker device 240, the marker element 240 has a “flower petal” shape having multiple protruding elements 310 (e.g., “petals”). Such configuration of the marker element 240, together with the softer polymeric tip 270, results in alternating sections 396, 398 at the distal end of the catheter 10, wherein the section 296 has more stiff marker material (from the marker element 240) for allowing the catheter 10 to resist ovalization, and the section 398 has more polymeric material (from the polymeric tip 270) for allowing the catheter 10 to achieve trackability.

The marker element 240 is also advantageous because it allows visualization under imaging, such as fluoroscopy, x-ray, CT, etc. The circumferentially alternating pattern achieved due to the protruding elements 310 allows visualization of torsional movement of the catheter. Also, in one implementation, the polymeric tip 270 extends less than 3 mm, or more preferably less than 2 mm, and even more preferably less than 1 mm, beyond the distal-most tip of the marker element 240. Such configuration may prevent, or at least reduce the risk of, collapsing of the polymeric tip 270 during aspiration and/or tracking. In addition, by having this “flush cut” design, a physician is able to know exactly where the catheter tip is during a medical procedure.

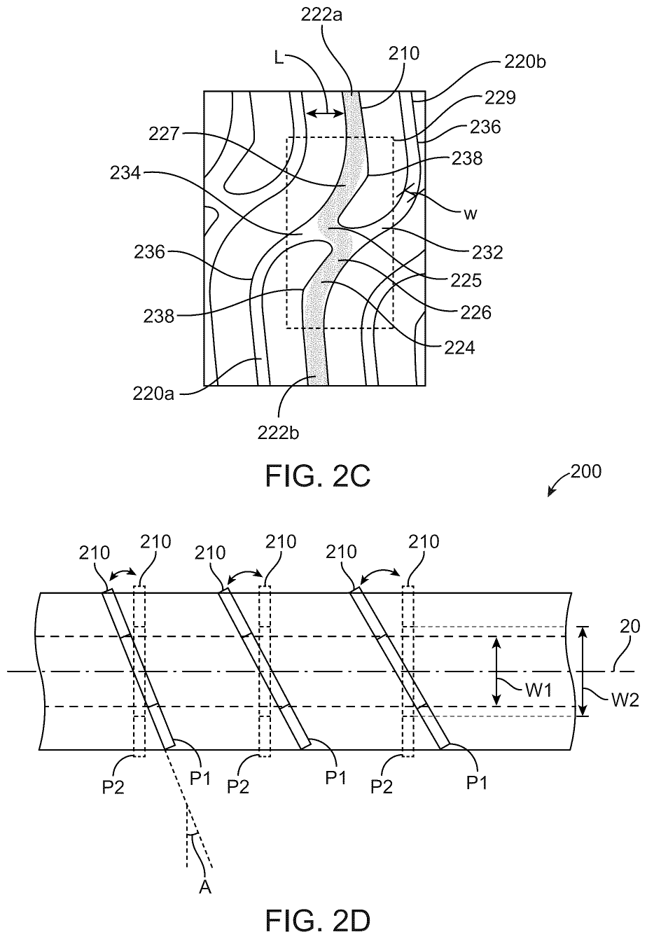

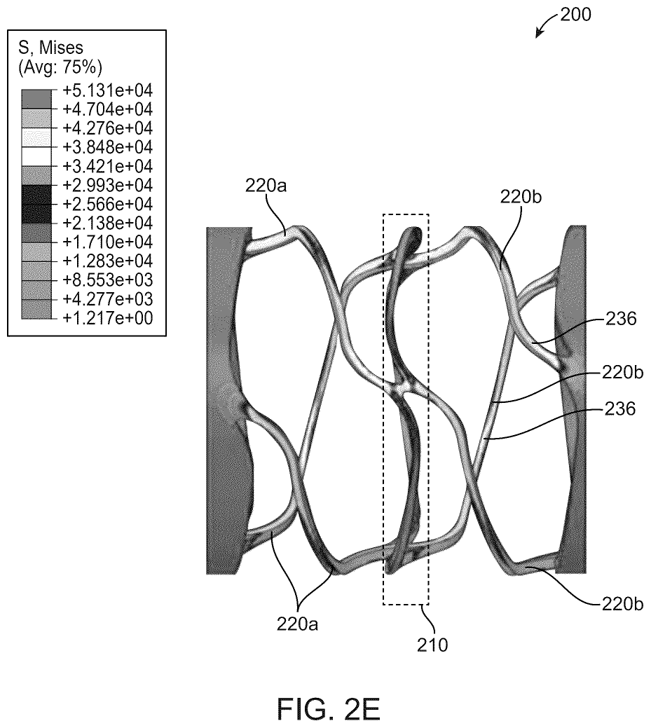

In some cases, the proximal end 302 of the ring structure 300 may optionally include tabs 400 disposed circumferentially around the axis 340 of the ring structure 300 (FIGS. 4-5). The tabs 400 are configured to be secured to the tubular structure 200, e.g., via weld (such as spot weld), adhesive, mechanical coupling, etc. The tabs 400 are advantageous because they provide a spacing between the marker element 240 and the tubular structure 200, which spacing allows for bending of the catheter 10. In the illustrated example, the marker element 240 has three tabs. In other cases, the marker element 240 may have two tabs, or more than three tabs.

In some cases, the ring structure 300 of the marker element 240 has a closed-loop configuration. In such cases, the ring structure 300 has a continuous wall extending circumferentially around the axis 340. In other cases, the ring structure 300 of the marker element 240 has an open-loop configuration. For example, the ring structure 300 may have a slit extending longitudinally from the distal end 301 to the proximal end 302. Such configuration may provide additional flexibility for the catheter 10.

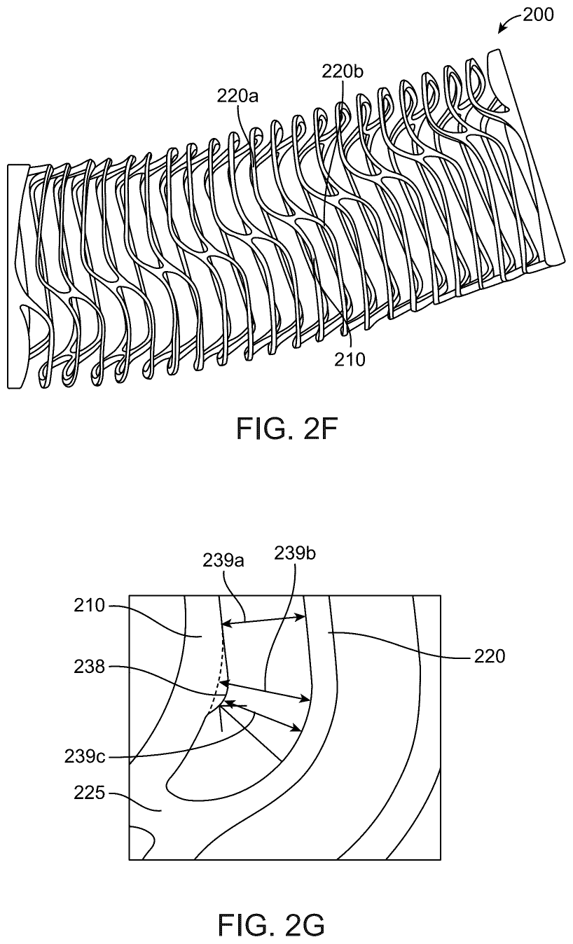

FIG. 6 illustrates a variation of the marker element 240 of FIG. 4, particularly showing the marker device having multiple openings 600. The openings 600 may be holes disposed circumferentially at the body 304 of the ring structure 300. The holes may be tiny through holes extending through a wall thickness of the ring structure 300. Each hole may be a circular hole, an elliptical hole, a square hole, a rectangular hole, or any hole with any geometric shape or any user-define shape. As shown in the figure, the openings 600 (holes) are distributed in both the longitudinal direction (i.e., the direction that is parallel to the longitudinal axis 340), and the circumferential direction (i.e., the direction extending circumferentially and being perpendicular to the longitudinal axis 340). In other cases, the marker element 240 may have only a single row of the openings 600 extending circumferentially around the axis 340. In such cases, the openings 600 are distributed circumferentially, and are not distributed longitudinally.

In one implementation, the openings 600 may be tiny holes having a cross-sectional dimension that is 1 mm or smaller. In other cases, each of the openings 600 may have a cross-sectional dimension that is larger than 1 mm. The openings 600 may be made using laser, punching, drilling, molding, etc. The openings 600 are advantageous because they allow material from the polymer layer 260 to anchor against the marker element 240. In some cases, the catheter 10 may include a second polymer layer (e.g., the polymer layer 262 of FIG. 8). In such cases, the openings 600 allow the first polymer layer 260 to bond to the second polymer layer 262, thereby preventing or reducing the risk of delamination (e.g., separation between the first and second polymer layers).

As described herein, the catheter 10 may include a first polymer layer (e.g., the polymer layer 260) is disposed over an exterior surface of the ring structure 300 and/or at least a part of the tubular structure 200. In such cases, parts of the first polymer layer 260 may extend into the openings 600 (e.g., holes) of the ring structure 300. The first polymer layer 260 may extend partly into the openings 600 of the ring structure 300, or may extend into the openings 600 completely through the wall thickness of the ring structure 300. The first polymer layer 260 may extend distally past a distal tip of the marker element 240 to form the polymeric tip 270, or to couple with the polymeric tip 270.

Also, in some cases, a second polymer layer 262 (shown in FIG. 8) may be disposed at an interior surface of the ring structure 300. The second polymer layer 262 may extend partly into the openings 600 of the ring structure 300. In such cases, material from the second polymer layer 262 may join and connect to the material from the first polymer layer 260 at a location that is inside the opening 600. Alternatively, the second polymer layer 262 may extend into the openings 600 completely through the wall thickness of the ring structure 300 to join and connect to the first polymer layer 260. In some cases, the second polymer layer 262 may extend distally past a distal tip of the marker element 240 to form the polymeric tip 270, or to couple with the polymeric tip 270.

In some cases, the ring structure 300 may be connected to the tubular structure 200 via a weld, or a connector, or an adhesive, etc. The ring structure 300 may surround a distal part of the tubular structure 200, and may secure to an exterior surface of the tubular structure 200. Alternatively, the ring structure 300 may be placed partially within the central lumen of the tubular structure 200, and may be secured to an inner surface of the tubular structure 200. In a further alternative technique, the proximal end of the ring structure 300 may face the distal end of the tubular structure 200 to radially align the wall of the ring structure 300 and the wall of the tubular structure 200. In other cases, the ring structure 300 may be formed integrally with the tubular structure 200. For example, the ring structure 300 and the tubular structure 200 may be molded together. Alternatively, the ring structure 300 and the tubular structure 200 (or at least a part of the tubular structure 200) may be formed together by obtaining a tube, and laser cutting the tube to form the ring structure 300 and the tubular structure 200.

### Polymeric Layers

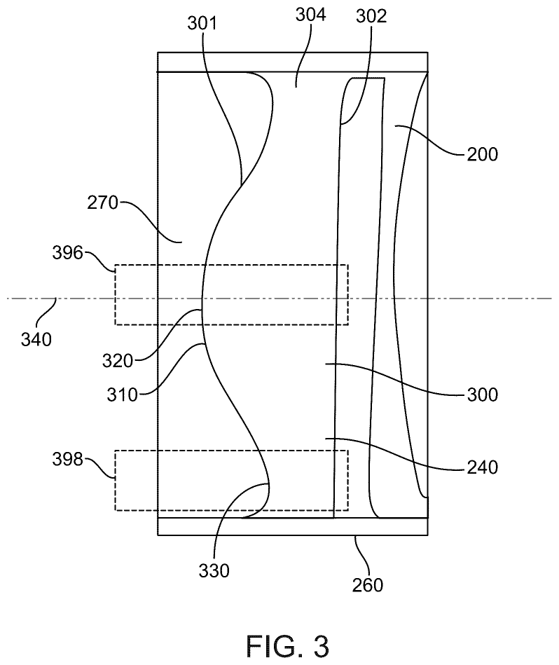

As discussed, in some cases, the catheter 10 may include a first polymeric layer 260 disposed circumferentially around the marker element 240 and/or at least a part of the tubular structure 200. FIG. 7 shows an example of a cross-section of the distal segment of the catheter 10. As shown in the figure, the catheter 10 includes the first polymeric layer 260 disposed circumferentially around an exterior surface of the ring structure 300 of the marker element 240. The first polymeric layer 260 extends proximally so that it also surrounds at least a part of the tubular structure 200. In some cases, the polymeric tip 270 is integral with the first polymeric layer 260. The polymeric tip 270 may be attached to the first polymeric layer 260 or may be formed together with the first polymeric layer 260.

As shown in FIG. 7, the catheter 10 further includes a second polymeric layer 262 disposed at an inner surface of the marker element 240 and an inner surface of the tubular structure 200. In some cases, the polymeric tip 270 is integral with the second polymeric layer 262. The polymeric tip 270 may be attached to the second polymeric layer 262 or may be formed together with the second polymeric layer 262.

In some cases, the first polymeric layer 260, the polymeric tip 270, and the second polymeric layer 262 may be integral with each other. In one implementation, the first polymeric layer 260, the polymeric tip 270, and the second polymeric layer 262 may be formed together. The first polymeric layer 260 and the second polymeric layer 262 may be made from a same material, or from different respective materials. Also, the polymeric tip 270 may be made from a same material as the first polymeric layer 260 and/or the second polymeric layer 270. In some cases, a first material 702 of the first polymeric layer 260 may extend to the space that is distal to the marker element 240 to form a first part of the polymeric tip 270, and a second material 704 of the second polymeric layer 262 may extend to the space that is distal to the marker element 240 to form a second part of the polymeric tip 270. In such cases, the polymeric tip 270 is made from the first material 702 of the first polymeric layer 260 and from the second material 704 of the second polymeric layer 262. The first material 702 and the second material 704 may be the same material, or may be different from each other.

The polymer layer 262 is advantageous because it provides a smooth inner lumen surface for the catheter 10. This allows another device (e.g., another catheter, a guidewire, a treatment device, a probe, etc.) to be inserted into the lumen of the catheter 10 and be moved relative to the catheter 10 with ease without rubbing against and/or without getting caught by components of the tubular structure 200 (which is exposed if the catheter 10 does not include the polymer layer 262). Also, providing both the polymer layer 260 and the polymer layer 262 is advantageous because they provide full encapsulation of the tubular structure 200. In some cases, the polymer layer 262 may be comprised of or coated with a hydrophilic coating to increase lubricity. In other cases, the catheter 10 may not include the second polymer layer 262. In such cases, the tubular structure 200 may have a smooth inner surface, and/or the components of the tubular structure 200 may have small gaps between them, to thereby allow another device to be moved relative to the catheter 10 with ease.

In the above examples, the catheter 10 is described as having the marker element 240. In other cases, the catheter 10 may not include the marker element 240. In such cases, the polymeric tube of the polymeric tip 270 may comprise a proximal tube end that abuts the distal end of the tubular structure 200.

In some cases, the first polymer layer 260 may comprise a first segment and a second segment proximal to the first segment. The first segment may be made from NEUSoft (e.g., NEUSoft UR862A (aka NEUSoft 62A), where “62A” refers to the hardness of the NEUSoft material). In some cases, the second segment 262 may be made from Pebax (e.g., Pebax 25D, Pebax 35D, or Pebax 45D). Also, in some cases, the first polymer layer 260 may include a third segment. The second segment may be made from a first Pebax, and the third segment may be made from a second Pebax that is different from the first Pebax. In further cases, the first polymer layer 260 may further comprise a fourth segment. The second segment may be made from a first Pebax, the third segment may be made from a second Pebax that is different from the first Pebax and the fourth segment may be made from a third Pebax that is different from the first Pebax and that is different from the second Pebax.

In some cases, the second polymer layer 262 may comprise a first segment and a second segment proximal to the first segment. The first segment of the second polymer layer 262 may be made from NEUSoft (e.g., NEUSoft 62A). The second segment of the second polymer layer 262 may be made from PTFE.

In some cases, the first segment of the first polymer layer 260 and a first segment of the second polymer layer 262 may be made from a same material (e.g., NEUSoft). The first segment of the first polymer layer 260 and the first segment of the second polymer layer 262 may be closer to a distal end of the catheter 10 than to a proximal end of the catheter 10.

Also, in some cases, a second segment of the first polymer layer 260 and a second segment of the second polymer layer 262 are made from different respective materials, wherein the second segment of the first polymer layer 260 is proximal to the first segment of the first polymer layer 260, and wherein the second segment of the second polymer layer 262 is proximal to the first segment of the second polymer layer 262.

FIG. 8 illustrates an example of the catheter 10 of FIG. 1, particularly showing the catheter 10 having the first polymer layer 260 and the second polymer layer 262. As shown in the figure, the first polymer layer 260 has a first segment 802 that is the distal-most segment. The first segment 802 is made from NEUSoft, such as NEUSoft UR862A (aka NEUSoft 62A) shown, but in other cases, the first segment 802 may be made from other NEUSoft materials (e.g., NEUSoft UR842A, NEUSoft UR852A, NEUSoft UR873A, etc.), or other non-NEUSoft materials. Using NEUSoft material (or another material with similar property as that of NEUSoft) for the distal most segment of the polymer layer 260 is advantageous because it has relatively low modulus of elasticity and high elasticity, which allows the polymer layer 260 to be highly elastic and flexible. As a result, after the catheter 10 has undergone high bending, the polymer layer 260 may elastically recover without being damaged and/or without being plastically deformed.

The first polymer layer 260 also includes a second segment 804 proximal to the first segment 802, wherein the second segment 804 is made from Pebax, such as Pebax 25D shown. In other cases, the second segment 804 of the first polymeric layer 260 may be made from other Pebax materials, or other non-Pebax materials. The first polymer layer 260 also includes a third segment 806 proximal to the second segment 804, wherein the third segment 806 is made from Pebax, such as Pebax 35D shown. In other cases, the third segment 806 of the first polymeric layer 260 may be made from other Pebax materials, or other non-Pebax materials. The first polymer layer 260 also includes a fourth segment 808 proximal to the third segment 806, wherein the fourth segment 808 is made from Pebax, such as Pebax 45D shown. In other cases, the fourth segment 808 of the first polymeric layer 260 may be made from other Pebax materials, or other non-Pebax materials. The first polymer layer 260 also includes a fifth segment 810 proximal to the fourth segment 808, wherein the fifth segment 810 is made from Coex comprised of Aesno and Pebax 45D. In other cases, the fifth segment 810 of the first polymeric layer 260 may be made from other materials.

It should be noted that the first polymer layer 260 is not limited to having five segments. In other cases, the first polymer layer 260 may have more than five segments, or fewer than five segments. In one implementation, the first polymer layer 260 may include only a single segment extending from the distal end to the proximal end.

As shown in FIG. 8, the second polymer layer 262 of the catheter 10 includes a first segment 852 that is the distal-most segment. The first segment 852 is made from NEUSoft, such as NEUSoft UR862A shown, but in other cases, the first segment 852 may be made from other NEUSoft materials (e.g., NEUSoft UR842A, NEUSoft UR852A, NEUSoft UR873A, etc.), or other non-NEUSoft materials. The second polymer layer 262 also includes a second segment 854 proximal to the first segment 852, wherein the second segment 854 is made from PTFE. In other cases, the second segment 854 of the second polymeric layer 262 may be made from other materials.

It should be noted that the second polymer layer 262 is not limited to having two segments. In other cases, the second polymer layer 262 may have more than two segments, or fewer than two segments. In one implementation, the second polymer layer 262 may include only a single segment extending from the distal end to the proximal end.

Also, in some cases, the first polymer layer 260 and the second polymer layer 260 may be made from the same material, such as NEUSoft, or any of other types of polymer.

In some cases, the first polymer layer 260 may be made from a material having an elastic modulus that is lower than that of the material of the tubular structure 200. Alternatively or additionally, the material of the first polymer layer 260 may be more elastic than that of the material of the tubular structure 200. Similarly, in some cases, the second polymer layer 262 may be made from a material having an elastic modulus that is lower than that of the material of the tubular structure 200.

Alternatively or additionally, the material of the second polymer layer 262 may be more elastic than that of the material of the tubular structure 200. As an example, each of the first polymer layer 260 and the second polymer layer 262 may have an elastic (Young's) modulus that is at least 2 Mpa and/or at most 7 Mpa, and/or a tensile elongation (tensile strain) at yield that is at least 300% (=3), and/or a tensile elongation (tensile strain) at break point that is at least 500% (=5). In some cases, each of the first polymer layer 260 and the second polymer layer 262 may have an elastic (Young's) modulus that is anywhere from 2 Mpa to 7 Mpa, and/or a tensile elongation (tensile strain) at yield that is anywhere from 300% to 1000%. Also in some cases, each of the first polymer layer 260 and the second polymer layer 262 may have a tensile strength of at least 6 Mpa, and/or a tensile strength of 70 Mpa or less.

In the illustrated example, the first segment (distal-most segment) of the first polymer layer 260 and the first segment (distal-most segment) of the second polymer layer 262 are made from the same material. This is advantageous because the same material of the first and second polymer layers 260, 262 provides homogenous material properties through the wall of the tubular structure 200, which may allow the polymer layers 260, 262 to be better connected to each other through the wall openings at the tubular structure 200. In other cases, the first segment (distal-most segment) of the first polymer layer 260 and the first segment (distal-most segment) of the second polymer layer 262 may be made from different respective materials.

During use, the tubular structure 200 may be undergoing compression, due to axial loading of the catheter 10, and/or bending of the catheter 10. As a result, the member (formed by the material of the polymer layer 260, the material of the polymer layer 262, the material of filler, or any combination of the foregoing) extending across the space 216 defined between components (e.g., between a ring element 210 and a connecting member 200) of the tubular structure 200 may be subjected to compression. Such member may be considered a polymeric member if it is composed of one or more polymers. To prevent the member from buckling (collapsing) during compression, the width of the space 216 (measured in the direction of compression) between the components of the tubular structure 200 may be designed so that nπ²EI/L²>F, where n is a factor accounting for end conditions of the member, E is the modulus of elasticity of the material of the member, I is the moment of inertia of the cross section of the member, and L is the column length of the member (which is the width of the space 216). Rewriting the above equation, the width L of the space 216 (wall gap) can be expressed as L< (nπ²EI/F)^{1/2}.

The factor n may be 4 assuming the end conditions of the member is “fixed” in rotation (i.e., the ends of the member are assumed not to rotate during compression). Alternatively, the factor n may be 1 assuming the end conditions of the member is “simple” (i.e., the ends of the member are allowed to rotate during compression), which will result in a more conservative determination for the column length L (the width of the space 216).

The elastic modulus E may be the elastic modulus of the material of the polymer layer 260, the elastic modulus of the material of the polymer layer 262, or the elastic modulus of the material of a filler in the space 216 that is different from the polymer layers 260, 262. In some cases, if the catheter 10 has all three items (i.e., the polymer layers 260, 262, and the filler between the polymer layers 260, 262) forming the member extending across the space 216, then the minimum elastic modulus (among the elastic moduli of the three items) may be selected for the above equation.

The moment of inertia I is the moment of inertial of the cross section of the member. In some cases, the moment of inertial I may be calculated based on the thickness t of the member and a width w of the member (i.e., assuming the cross section has a rectangular shape with a thickness t and a width w). In particular, the moment of inertial I for a rectangle is wt³/12.

F is the maximum compression force experienced by the member during use. In some cases, F can be determined by computer modeling, or by testing a prototype.

Configuring the width of the space 216 based on the above parameters is advantageous because it enhances compression resistance for the catheter 10, and allows the catheter 10 to undergo bending with higher curvature (i.e., lower radius of curvature).

In some cases, the width of the space 216 may be a value that is anywhere from 100 microns to 400 microns, and more preferably anywhere from 120 microns to 300 microns, and more preferably anywhere from 140 microns to 280 microns, and more preferably anywhere from 160 microns to 260 microns. In other cases, the space 216 may be more than 400 microns, or less than 100 microns.

Also, it should be noted that the width L of the space 216 depends on the elastic modulus E. If a material with higher E is chosen, then the width L of the space 216 may be longer. Rewriting the above equation: L²<nπ²EI/F. Then F/(nπ²I)<E/L². Accordingly, the material and the width L of the space 216 (wall gap) may be designed so that E/L²>F/(nπ²I). In some cases, the E/L may be anywhere between 20 to 400 N/mm∧4, and more preferably anywhere between 25 to 360 N/mm∧4, and even more preferably anywhere between 28 to 300 N/mm∧4, and even more preferably anywhere between 30 to 280 N/mm∧4.

Also, in some cases, the space 216 (wall gap) may be defined as the shortest distance between adjacent elements of the tubular structure 200, such as between a ring element 210 and a connecting member 220. In some embodiments, the space 216 may be equal to, or greater than, a width of the ring element 210. The width of the ring element 210 may be defined as a thickness of the ring element 210 measured in a direction that is parallel to (or that forms an acute angle with) the longitudinal axis 20, and/or in a direction that is perpendicular to a radial direction with respect to a cross-section of the tubular structure 200. An example of the width Wr of the ring element 210 is shown in FIG. 2B. Alternatively or additionally, the space 216 may be equal to, or greater than, a width of the connecting member 220. The width of the connecting member 220 may be defined as a thickness of the connecting member 220 measured in a direction that is parallel to (or that forms an acute angle with) the longitudinal axis 20, and/or in a direction that is perpendicular to a radial direction with respect to a cross-section of the tubular structure 200. An example of the width Wc of the connecting member 220 is shown in FIG. 2B. In some cases, the width of the ring element 210/connecting member 220 may be considered a kerf width (as that is the material remaining after parts of a tube is removed to create the tubular structure 200 with the ring elements 210 and connecting members 220). Thus, Wr and Wc may be considered as examples of a kerf width. In some embodiments, the ratio calculated as the space 216 divided by the kerf width may be anywhere from 1 to 5 (e.g., at least 3, or 4, etc.). This ensures that there is sufficient material in the wall gap to connect the layers 260, 262 forming a laminated structure, and/or to provide good compression resistance.

### Tube Manufacturing Process

Various techniques may be employed to make a tube having the tubular structure 200 and the polymer layer(s) (e.g., layer 260 and/or layer 262) for the catheter 10. In some cases, a coating material may be applied so that the material is disposed in the openings of the tubular structure 200 and forms an outer layer covering the exterior surface of the tubular structure 200. The coated tubular structure 200 forms the tube. In other cases, the coating material may encapsulate the tubular structure 200 to form the tube. In such cases, the coating material covers the exterior surface of the tubular structure, covers the interior surface of the tubular structure, as well as filling the openings (e.g., slots) through the wall of the tubular structure 200.

In some cases, the coating material forming part of the tube may have a modulus of elasticity that is lower than the modulus of elasticity of the tubular structure 200. For example, the coating material may have a modulus of elasticity that is less than 50%, or more preferably less than 30%, or more preferably less than 20%, or more preferably less than 10%, or more preferably less than 5%, or more preferably less than 1%, of that of the tubular structure 200. In one implementation, the coating material may have a modulus of elasticity that is less than 15 Mpa (e.g., 10 Mpa or less).

Also, the coating material forming part of the tube may have the ability to undergo significant elongation before yielding. For example, in some embodiments, the coating material may be capable of having a strain (defined as an amount of elongation of the material divided by the length of the material) of at least: 20%, 40%, 60%, 80%, 100%, 200%, 300%, 400%, 500%, 600%, 700%, 800%, 900%, 1000%, or greater.

Various materials may be utilized as the coating material. By means of non-limiting examples, the coating material may include polyurethane, polyurethane-based material, silicon-based material, any material having polyurethane dispersion or silicon-based dispersion, etc. Examples of coating material that may be used include CD102® or AD111® from Covestro, Gelest Ex-sil50® from Gelest, NEUSoft (e.g., NEUSoft 42A, NEUSoft 52A, NEUSoft 62A, NEUSoft 73A, etc.), Pebax (e.g., Pebax 25D, Pebax 35D, or Pebax 45D, etc.), etc.

In some embodiments, the tubular structure 200 is configured to be significantly much more flexible compared to the polymeric tubing (formed by the layer 260 and/or the layer 262). In such cases, the resulting tube will have one or more mechanical properties that are contributed predominantly (e.g., more than 50%, more than 60%, more than 70%, more than 80%, more than 90%, etc.) by the polymeric tubing. By means of non-limiting examples, the one or more mechanical properties may be a bending stiffness, an axial stiffness, a torsional stiffness, a shear stiffness, or any combination of the foregoing. Also, in some cases, the stiffness of the resulting tube formed by the polymeric tubing (comprised of the layer 260 and/or the layer 262) and the tubular structure 200 may be at least 4 times, and more preferably at least 8 times, and more preferably at least 12 times, and more preferably at least 16 times, the stiffness of the tubular structure 200 alone (i.e., without the polymeric tubing). In some embodiments, the material(s) of the polymeric tubing has a lower elastic modulus and is more elastic compared to the material of the tubular structure 200. Despite of this, the resulting tube formed by the polymeric tubing and the tubular structure 200 is at least 4 times the stiffness of the tubular structure 200 alone.

In one or more embodiments, the tube may optionally further include a hydrophilic coating that is disposed on the exterior surface of the tube, and/or on the interior surface of the tube. In some cases, an initial coating material may first be applied on the tubular structure 200 to fill the openings at the wall of the tubular structure 200, and optionally to cover the exterior and/or interior surface of the tubular structure 200. Then the hydrophilic coating is applied over the initial coating material.

Constructing the tube using the tubular structure 200 (providing a majority of the mechanical properties), and using the very soft coating material, is advantageous because it prevents the distal end of the tube from being too stiff, and because it allows design of catheter to be easier with resulting behavior of the catheter being more predictable (because computational modeling of the catheter may be made based on the tubular structure design only).

Various techniques may be employed to apply the coating material to the tubular structure 200. For example, in some cases, polymer extrusion(s) may be applied/assembled to the inside surface and/or outside surface of the tubular structure 200, and laminated to reflow around it, thereby encapsulating the tubular structure 200. In one implementation, after polymer extrusions may be applied to both the inside surface and outside surface of the tubular structure 200. The polymer extrusions are then heated to cause polymeric materials to fill the gaps at the wall of the tubular structure 200, thereby resulting in a laminated structure in which the polymer layers on opposite sides of the wall of the tubular structure 200 are connected to each other through the gaps at the wall of the tubular structure 200, and encapsulating the tubular structure 200.

In some cases, a deposition technique may be employed to deposit the coating material onto the tubular structure 200. In other cases, a dipping process may be utilized to apply the coating material onto the tubular structure 200.

In some cases, when using a dipping technique to apply the coating material onto the tubular structure 200, a barrier may be provided inside the central lumen of the tubular structure 200 to prevent the coating material from entering into the central lumen. For example, in some embodiments, the interior surface of the tubular structure 200 may be masked to prevent the coating material from outside the tubular structure 200 from flowing into the central lumen through the openings at the wall of the tubular structure 200. In other embodiments, a tube or rod may be placed inside the central lumen of the tubular structure 200 to act a barrier for preventing the coating material from entering into the central lumen. The tube or the rod may be made from PTFE, HDPE, stainless steel, or any of other suitable materials.

The tubular structure 200 may then be placed into a reservoir of the coating material in liquid or viscous form. The coating material may be tailored to have certain specific viscosity to assist with the coating process. In some cases, the tubular structure 200 may be placed vertically into the reservoir with the longitudinal axis of the tubular structure 200 forming an angle that is 90°+/−25° with respect to a top surface of the liquid in the reservoir. In other cases, the tubular structure 200 may be placed horizontally into the reservoir with the longitudinal axis of the tubular structure 200 forming an angle that is 0°+/−25° with respect to a top surface of the liquid. In other cases, the tubular structure 200 may be placed into the reservoir at other angles that are different from the above examples.

In some cases, the tubular structure 200 may be inserted into the reservoir at a certain rate, such as anywhere from 0.01 cm/sec to 5 cm/sec. In other cases, the tubular structure 200 may be inserted at a rate that is slower or faster than the above range of rates. Also, in some cases, as the tubular structure 200 is inserted into the reservoir, the tubular structure 200 may be rotated at a certain rate, such as 2-10 rotations per minute. The rotational speed may be slower or faster than 2-10 rotations per minute in other cases. In further cases, the tubular structure 200 may not be rotated as it is being inserted into the reservoir.

After the tubular structure 200 is dipped into the reservoir of the coating material, the tubular structure 200 may then be removed from the reservoir. In some cases, the tubular structure 200 may be removed from the reservoir at a certain rate, such as anywhere from 0.01 cm/see to 10 cm/sec. In other cases, the tubular structure 200 may be removed from the reservoir at a rate that is slower or faster than the above range of rates. Also, in some cases, as the tubular structure 200 is removed from the reservoir, the tubular structure 200 may be rotated at a certain rate, such as 2-10 rotations per minute. The rotational speed may be slower or faster than 2-10 rotations per minute in other embodiments. In further cases, the tubular structure 200 may not be rotated as it is being removed from the reservoir of the coating material.

As a result of dipping the tubular structure 200 into the reservoir and removing it from the reservoir, a first layer (e.g., layer 260) of coating material is disposed on the exterior surface of the tubular structure 200. The coating material also spans and fills up the openings at the wall of the tubular structure 200.

After the tubular structure 200 is removed from the reservoir, the tubular structure 200 with the coating material is held at certain time and at certain temperature to solidify the coating material. The time for the coating material to solidify may be anywhere from 1 minute to 120 minutes. In other embodiments, the solidifying time may be faster than 1 minute, or longer than 120 minutes. Also, in some cases, the temperature for solidifying the coating material may be anywhere from 20° C. to 100° C. In other cases, the temperature for solidifying the coating material may be less than 20° C. or higher than 100° C.

In some cases, the insertion of the tubular structure 200 into the reservoir, and the removing of the tubular structure 200 from the reservoir, may be repeated one or more times (e.g., anywhere from 1 additional time to 30 additional times, or more) until a desired thickness for the coating material is achieved. In some cases, the thickness of the coating material created on the exterior surface of the tubular structure 200 may be anywhere from 0.0001 inch to 0.003 inch, or greater.

In some cases, the applying of the coating material onto the tubular structure 200 may be performed in a vacuum. For example, in some cases, the reservoir of coating material may be placed in a vacuum chamber, and the dipping and removing of the tubular structure 200 may be performed inside the vacuum chamber. The solidifying of the coating material may also occur inside the vacuum chamber.

In some cases, after the coating material is solidified, a hydrophilic coating may be applied on the solidified coating. The application of the hydrophilic coating may be performed using a dipping technique that is similar to that described above.

In other cases, the hydrophilic coating may be applied on the solidified coating using a deposition technique. The hydrophilic coating may be applied to the exterior surface of the tube 11 and/or to the interior surface of the tube 11. In some cases, the hydrophilic coating may be considered to be a part of the tube 11.

It should be noted that the processing of coating the tubular structure 200 is not limited to the examples described above, and that the tubular structure 200 may be coated using other techniques, or variations of the techniques described. For example, in other cases, a barrier may not be provided inside the central lumen of the tubular structure to prevent the coating material from entering into the central lumen. Instead, coating material is allowed to flow into the central lumen during the dipping process. In such case, after the tubular structure 200 is removed from the reservoir, and before the coating material in the central lumen of the tubular structure 200 solidifies, a plunger may be placed inside the central lumen and be moved longitudinally through the tubular structure 200 to remove excess material inside the central lumen. In some cases, all coating material inside the central lumen is removed so that there is no coating material disposed on the interior surface of the tubular structure 200. In other cases, some but not all of the coating material inside the central lumen is removed so that a layer (e.g., layer 262) of the coating material remains on the interior surface of the tubular structure 200. In further embodiments, the removing of the excess coating material inside the central lumen of the tubular structure 200 may be removed using a cutter after the coating material has been solidified.

In other cases, before the tubular structure 200 is inserted into the reservoir of coating material, a rod or a tube (smaller than a size of the central lumen of the tubular structure 200) may be placed inside the central lumen of the tubular structure 200. The rod or the tube has an exterior surface that is spaced away from the inner surface of the tubular structure 200. This allows the coating material to fill the space between the rod/tube and the interior surface of the tubular structure 200, thereby creating a layer (e.g., layer 262) of coating on the interior surface of the tubular structure 200. The coating material also fills the openings at the wall of the tubular structure 200, and extends to outside the tubular structure 200 to create a layer (e.g., layer 260) of coating on the exterior surface of the tubular structure 200.

In further cases, instead of using the dipping technique, the coating material may be pumped to encapsulate the tubular structure 200 at certain flow rates to create a desired thickness of the coating.

### Method of Use

The catheter 10 described herein may be any type of catheter in different embodiments. For example, the catheter 10 may be an implant delivery catheter configured to deliver an implant, such as to treat aneurysm. As another example, the catheter 10 may be a guide catheter configured to be delivered over a guidewire, and also configured to deliver a medical tool. As further examples, the catheter 10 may be a drug delivery catheter configured to deliver a drug, or a biopsy catheter configured to transport tissue collected from inside a patient.

In some embodiments, the catheter 10 described herein may be utilized to accessed a vasculature inside a patient. During use, the distal end of the catheter 10 is inserted into a blood vessel of the patient, and is advanced distally inside the blood vessel. In some cases, a guidewire may be already delivered inside the patient, and advanced to reach a target site. In such cases, the catheter 10 may be advanced over the guidewire, until the distal end of the catheter 10 reaches the target site. Alternatively or additionally, a guide-catheter may be inserted into the blood vessel first, and may be advanced to a desired location inside the patient's body. Then, the catheter 10 may be placed inside the guide-catheter and may be advanced in the guide-catheter until the distal end of the catheter 10 reaches a target site.

Also, in some embodiments, the catheter 10 may be used to facilitate an insertion and guidance of interventional devices into a selected blood vessel in the neurovascular system, or to facilitate the removal of thrombus and revascularization of blood vessels in patients experiencing an acute ischemic stroke. The catheter 10 may first be hydrated and flushed, and may then be advanced into the lumen of a guide-catheter that is already inserted inside the patient. The catheter 10 is advanced until the tip of the catheter 10 reaches a target location. In some cases, while the catheter 10 is inside the guide-catheter, the catheter 10 may include a neurointerventional guidewire and/or a delivery assist catheter in its lumen.

Alternatively, if a wire (e.g., guidewire) and/or delivery assist catheter is used, the tip of the wire and/or the tip of the delivery assist catheter may be advanced first, and may be rotated to direct the tip orientation toward an intended vessel. The catheter may then be advanced over the wire and/or over the delivery assist catheter. There may be occasions where the catheter 10 can be advanced directly into the target vessel without the use of a guide catheter and/or without the use of a wire (e.g., guidewire). In such cases, the catheter 10 may or may not include a wire (e.g., guidewire, such as neurointerventional guidewire) and/or a delivery assist catheter in its lumen.

As the catheter 10 is being navigated through the vasculature, the catheter may undergo bending, tension, and compression. When these happen, adjacent ring elements 210 will move relative to each other. In particular, when the catheter 10 is under tension, the adjacent ring elements 210 will move away from each other. When the catheter 10 is under compression, the adjacent ring elements 210 will move towards each other. When the catheter 10 is being bent, the adjacent ring elements 210 will rotate relative to each other. Due to the auxetic angle of the ring elements 210, as the catheter 10 is undergoing tension and bending, the lumen of the catheter will not collapse as described herein.

Also, as the adjacent ring elements 210 move relative to each other, the connecting members 220 between the adjacent ring elements 210 will move relative to the adjacent ring elements 210. As a result, the angle between the connecting member 220 and the ring element 210 (to which the connecting member 220 is attached) will change—i.e., will decrease or increase. The connecting members 220 transfer stresses between the adjacent ring elements 210 and maintains the structural integrity for the tubular structure 200. If the width (measured along a direction that is perpendicular to a radial axis of the tubular structure 200) of the connecting member 220 is less than the thickness (measured along a radial direction of the tubular structure 200), the movement of the connecting member 220 will be in a direction that is perpendicular to the radial axis of the tubular structure 200 (e.g., along a tangential direction with respect to a cross-section of the tubular structure 200). This has the benefit of preventing the connecting member 220 from moving radially (e.g., inwardly toward the lumen of the catheter 10, or outwardly toward the exterior surface of the catheter 10).

In addition, while the catheter 10 is being navigated inside the blood vessel, the polymeric materials (fillers) in the spacing among the ring elements 210 and the connecting members 220 transfer stress among the ring elements 210 and the connecting members 220. In some cases, the transferred stress may be compression stress. In such cases, the polymeric fillers will not buckle in response to the compression due to the length of the polymeric filler (which is equal to the width of the spacing between the ring element 210 and the connecting member 220) being configured based on buckling parameter(s) described herein.

After the catheter 10 is desirably positioned inside the patient, the catheter may then be utilized to one or more items to treat a medical condition. For example, the catheter 10 may deliver an implant, such as to treat aneurysm. As another example, the catheter 10 may deliver a drug to treat a medical condition inside the patient. As a further example, the catheter 10 may transport a device, such as a guidewire, a treatment device, a diagnostic tool, etc. In other embodiments, the catheter 10 may transport tissue collected from inside a patient. The polymeric layer disposed inside the inner wall surface of the tubular structure 200 provides a smooth surface that allows the item(s) in the lumen of the catheter 10 to be transported easily.

As used in this specification, the term “relaxed state” (e.g., a relaxed state of the tubular structure, a relaxed state of the catheter, etc.) refers to a state of an object in which no external force is applied against the object (other than the force due to gravity). For example, a relaxed state of a tubular structure/a catheter may refer to a state of the tubular structure/catheter that is placed on a surface without having bending force, axial force, and torsional force applied to the tubular structure/catheter.

Although particular embodiments have been shown and described, it will be understood that it is not intended to limit the claimed inventions to the preferred embodiments, and it will be obvious to those skilled in the art that various changes and modifications (e.g., the dimensions and/or shapes of various parts) may be made without department from the spirit and scope of the claimed inventions. The specification and drawings are, accordingly, to be regarded in an illustrative rather than restrictive sense. The claimed inventions are intended to cover alternatives, modifications, and equivalents.

What is claimed is:

1. A catheter, comprising:
a tubular structure having a first ring element, a second ring element, and a third ring element;
wherein the tubular structure comprises a first set of connecting members between the first ring element and the second ring element;
wherein the tubular structure comprises a second set of connecting members between the second ring element and the third ring element;
wherein the second ring element comprises a first ring portion and a second ring portion, wherein each of the first and second ring portions extends in a nonperpendicular direction with respect to a longitudinal axis of the tubular structure, and wherein the first ring portion and the second ring portion together form a non-planar configuration for the second ring element; and
wherein each of the first ring portion and the second ring portion extends at least 18% of a circumference of the second ring element.

2. The catheter of claim 1, wherein the first ring portion and the second ring portion are different respective circumferential parts of the second ring element.

3. The catheter of claim 1, wherein the first ring portion has a first flat part lying in a first plane, wherein the second ring portion has a second flat part lying in a second plane that is different from the first plane, and wherein each of the first plane and the second plane forms a non-perpendicular angle with respect to a longitudinal axis of the catheter.

4. The catheter of claim 1, wherein the second ring element also comprises a joint portion between the first ring portion and the second ring portion, wherein the joint portion comprises a central portion, a first portion, and a second portion, and wherein the first and the second portions are on opposite sides of the central portion.

5. The catheter of claim 4, wherein the first portion and the second portion of the joint portion are curving in respective opposite directions.

6. The catheter of claim 4, wherein the central portion of the joint portion is configured to rotate and/or to resist a moment when the catheter is being tensioned or is undergoing bending.

7. The catheter of claim 1, wherein the first set of connecting members and the second set of connecting members are configured to move relative to the second ring element during bending and/or axial loading of the catheter.

8. The catheter of claim 1, wherein one of the connecting members in the first set has a width and a thickness, and wherein the width is less than the thickness.

9. The catheter of claim 1, wherein a total number N of the connecting members in the first set meets the condition N>F/(S*A), where F is an axial load on the catheter, S is an allowable axial stress on each of the connecting members in the first set, and A is an area of a cross section of one of the connecting members.

10. The catheter of claim 1, wherein a space between one of the connecting members in the first set and the second ring element has a width L, and wherein the width L of the space meets the condition L<(mr2E I/F)1/2, where n=4 or lower, E is an elastic modulus of a material of a polymeric structure spanning the space, F is an axial loading on the polymeric structure, and I is a moment of inertia of a cross section of the polymeric structure.

11. The catheter of claim 1, wherein a space between one of the connecting members in the first set and the second ring element has a width L, wherein the catheter further comprises a polymeric structure spanning the space, the polymeric structure having an elastic modulus E, and wherein a ratio E/L2 meets the below condition: F/(mr2I)<E/L2, where F is an axial loading on the polymeric structure, n=4 or lower, and I is a moment of inertia of a cross section of the polymeric structure.

12. The catheter of claim 1, further comprising a marker element attached to or extending from a distal end of the tubular structure.

13. The catheter of claim 12, further comprising a polymer layer coupled to the tubular structure, wherein the polymer layer extends distally past a distal tip of the marker element to form a polymeric tip, wherein the polymeric tip extends less than 3 mm beyond the distal tip of the marker element.

14. The catheter of claim 1, further comprising a first polymer layer disposed circumferentially around an exterior surface of the tubular structure.

15. The catheter of claim 14, wherein the first polymer layer has an elastic modulus that is at least 2 Mpa and/or at most 7 Mpa, a tensile elongation at yield that is at least 300%, a tensile elongation at break that is at least 500%, or any combination of the foregoing.

16. The catheter of claim 14, further comprising a second polymer layer disposed at an interior surface of the tubular structure.

17. The catheter of claim 16, wherein the second polymer layer has an elastic modulus that is at least 2 Mpa and/or at most 7 Mpa, a tensile elongation at yield that is at least 300%, a tensile elongation at break that is at least 500%, or any combination of the foregoing.

18. The catheter of claim 16, wherein the first polymer layer and the second polymer layer form a tubing encapsulating the tubular structure; and
wherein a material of the tubing has a modulus elasticity that is higher than a material of the tubular structure, and is more elastic than the material of the tubular structure.

19. The catheter of claim 18, wherein a stiffness of a combination of the tubular structure and the tubing is at least four times a stiffness of the tubular structure alone.

20. The catheter of claim 1, wherein the tubular structure has a wall gap to kerf width ratio that is anywhere from 1 to 5.
